(19) United States
(12) Patent Application Publication
(10) Pub. No.: US 2003/0163142 A1
(43) Pub. Date: Aug. 28, 2003

(54) System and method for guiding the movements of a device to a target particularly for medical applications

(76) Inventors: Yoav Paltieli, Haifa (IL); Victor Segalescu, Haifa (IL); Ron Nagar, Haifa (IL); Michael hareven, Haifa (IL)

Correspondence Address: Eitan, Pearl, Latzer & Cohen Zedek, LLP., Suite 1001, 10 Rockefeller Plaza, New York, NY, 10020, US

(21) Appl. No.: 10/336,039

(22) Filed: Jan. 3, 2003

Related U.S. Application Data
(63) Continuation of application No. 09/555,364, filed on May 30, 2000.
Application No. 09/555,364 is a 371 of international application No. PCT/IL98/00578, filed on Nov. 26, 1998.

(30) Foreign Application Priority Data
Nov. 27, 1997 (IL) 122336

(51) Int. Cl. A61B 19/00

(52) U.S. Cl. 606/130

(57) ABSTRACT

A system and method for guiding the movements of a device to a target particularly for medical applications is provided. In one aspect of the invention, the system guides the movements of a device toward a target, which is viewable from an image. The system includes at least one scanning apparatus for creating the image including the target, at least one transmitter at a first reference location for transmitting radiant energy from the reference location, several position sensors and a data processor. The position sensors include at least one position sensor on the device for receiving the radiant energy, at least one position sensor on the scanning apparatus for receiving the radiant energy and at least one position sensor for receiving the radiant energy on either the device or the scanning apparatus. The data processor is in operative communication with the transmitter and each of the position sensors, for monitoring the accuracy of the calculated position of the device with respect to the scanning apparatus.

### PRIOR APPLICATION DATA

The present application is a continuation of prior U.S. application Ser. No. 09/555,364, filed May 30, 2000 and entitled “SYSTEM AND METHOD FOR GUIDING THE MOVEMENTS OF A DEVICE TO A TARGET PARTICULARLY FOR MEDICAL APPLICATIONS” incorporated herein by reference.

### FIELD OF THE INVENTION

The present invention relates to guidance systems and safety systems associated therewith, and also to methods for their use for guiding the movements of a device towards a target. The invention is particularly useful in medical applications for guiding the movements of a medical device, such as an aspiration, biopsy needle, or an endoscope, through biological tissue in a body to a target therein, in conjunction with an imaging system, such as ultrasound, CT or MRI.

### BACKGROUND OF THE INVENTION

Guiding systems are used in medical applications for the purpose of guiding various types of medical devices, such as aspiration and biopsy needles, endoscopes, etc., towards specific targets within the body. For example, where guiding a biopsy needle, a position sensor is usually rigidly affixed to the top of the needle, and its absolute position in space is determined. The part of the body in which the target tissue is located is usually imaged. by an imaging system, such as ultrasound, CT, MRI. The absolute. position of the plane displayed by the imaging system can likewise be determined with the aid of a similar position sensor, so that the relative location and orientation of the needle with respect to the target tissue can be calculated. Once these values are determined, it is possible to compute the expected path of the needle towards the target and to display it on the image in order to enable the physician to navigate the biopsy needle (or other medical devices) to the target. Such a composite device using ultrasound imaging for guidance has been described in our Patent Application No. PCT/IL96/00050, published Feb. 6, 1997, the description of which is hereby incorporated by reference. In that case, the location and orientation of the ultrasound scan plane is measured by a sensor mounted on the ultrasound transducer.

The most prevalent method for effecting the required measurements is by transmitting a set of signals from a transmitter located at a fixed location in space, which signals are received by a set of sensors mounted on the biopsy needle (or other device being navigated). Both the transmitter and the receiver are linked, in this case, to a separate set of Cartesian coordinates. The relative position and orientation of the receiver Cartesian coordinates with respect to the transmitter ones is given by a translation vector pointing from the transmitter's to the receiver's origin of coordinates and by a three-dimensional rotation matrix. The transmitter signals received by the receivers are sufficient to determine the components of the said vector and matrix. Thus, the location and orientation of the device can be calculated with respect to the transmitter's coordinates. The signals utilized for this purpose may be light, radio frequency (RF) or low-frequency (LF) electromagnetic waves, etc.

The above method is sensitive to measurement errors caused by interfering objects and/or interfering electromagnetic fields present in the vicinity of the receiving sensors. In the case of medical intervention procedures, such measurement errors may cause irreversible damages to the patient's body, and as such must be avoided.

### OBJECTS AND BRIEF SUMMARY OF THE PRESENT INVENTION

An object of the present invention is to provide systems, and also methods, having advantages in the above respects for guiding the movements of a device towards a target. More particularly, an object of the present invention is to provide systems and methods that may be used for guiding medical devices towards a target in a body, which method and system will produce an indication, more particularly will actuate an alarm, if interfering objects and/or interfering electromagnetic fields are present which might produce measurement errors that could cause serious injury to the patient's body.

According to one aspect of the present invention, there is provided a system for guiding the movements of a device towards a target, comprising: transmitter apparatus at a reference location in space for transmitting radiant energy from said reference location; position sensor(s) on said device to be guided to the target, said position sensor(s) receiving radiant energy from the transmitter apparatus and producing an output corresponding to the position of said device with respect to said reference location in space; and a data processor receiving the output of said position sensor and calculating the position of said device with respect to said reference location; position sensor(s) on said scanning device, which is scanning the target, as said position sensor(s) receiving radiant energy from the transmitter apparatus and producing an output corresponding to the position of said scanning device with respect to said reference location in space; and a data processor receiving the output of said position sensor(s) and calculating the position of said device with respect to said reference location and also calculating the position of said guided device with respect to said scanning device/imaging plane. Such a system was described in our Patent Application No. PCT/IL96/00050, incorporated by reference in its entirety herein, and further referred as first guidance system.

According to a further aspect of the invention the guiding system is employed with a safety system characterized in that transmitter apparatus includes a first transmitter (T₁) and a second transmitter (T₂) in a known spatial relationship to each other defined by a known vector ( [symbol] ₁₂); and further characterized in that said data processor:

(a) produces a measurement of the location and orientation of the position sensor with respect to said first transmitter (T₁) and defines same by a first vector;

(b) produces a measurement of the location and orientation of the position sensor with respect to said second transmitter (T₂) and defines same by a second vector;

(c) calculates from said first vector and the said second vector a measured vector (\vec{T}₁₂)ₘ between the two transmitters;

(d) compares the measured vector (\overline{T}₁₂)ₘ with the known vector (\overline{T}₁₂); and

(e) produces an output corresponding to the absolute value of the difference between the measured vector (\overline{T}₁₂)ₘ and the known vector (\vec{T}₁₂).

According to further features in the described preferred embodiment, the data processor includes an alarm, compares the output produced in step (e) with a predetermined threshold value, and actuates said alarm if the output produced in step (e) exceeds said predetermined threshold value.

According to still further features in the described preferred embodiment, the device is a medical device, more particularly an interventional device such as a biopsy needle, to be guided through the body to a target therein. An imaging system is provided which scans the body by a scanning device along a plurality of scan lines. The scanning device also includes a second position sensor receiving radiant energy from the first and second transmitters. The data processor produces an output corresponding to the position of the second position sensor, and thereby of the scanning device, with respect to said known location in space. is The data processor performs said step (a)-(e) also with respect to said second position sensor to produce an output error with respect to the measured vector (\overline{T}₁₂)ₘ for the scanner, and utilizes the larger of said two output errors for comparison with the predetermined threshold and for activating the alarm.

According to further features in the described preferred embodiment, the scanning device is an ultrasound scanner (transducer) and the first and second transmitters are mounted at the opposite ends of an arm having a known length and orientation in space defining the known vector (\overline{T}₁₂).

The invention also provides methods for guiding the movements of a device towards a target.

The invention also provides apparatus and methods for ensuring the safety of the system by providing redundant transmitters, redundant receivers and/or alternating signaling mechanisms.

The invention also discloses additional guidance systems and apparatus and methods for ensuring the safety of these systems by providing redundant transmitters, redundant receivers and/or alternating signaling mechanisms.

More particularly, an object of the present invention is to provide systems and methods that may be used for guiding medical devices towards a target in a body, which method and system will produce an indication, more particularly will actuate an alarm, if interfering objects and/or interfering electromagnetic fields are present which might produce measurement errors that could cause serious injury to the patient's body.

Further features and advantages of the invention will be apparent from a description below.

### BRIEF DESCRIPTION OF THE DRAWINGS

The invention is herein described, by way of example only, with reference to the accompanying drawings, wherein like reference numerals or characters indicate corresponding or like components. In the drawings:

FIG. 1 pictorially illustrates one form of a system constructed in accordance with the present invention for guiding the movements of a device by ultrasound, in this case a medical biopsy needle, towards a target, employing a safety system based on redundant transmitters, where the relative positional relationship between the transmitters is known;

FIG. 2 diagrammatically illustrates the various vectors involved in using the system of FIG. 1 for guiding the movements of the biopsy needle towards the target;

FIG. 3a is a flowchart illustrating Safety Algorithm 11 for the operation of the systems of the present invention;

FIG. 3b is a flowchart illustrating Safety Algorithm 12 for the operation of the systems of the present invention;

FIG. 3c is a flowchart illustrating Safety Algorithm 13 for the operation of the systems of the present invention;

FIG. 3d is a flowchart illustrating Safety Algorithm 14 for the operation of the systems of the present invention;

FIG. 3e is a flowchart illustrating the sequential analysis that can be performed on the output of Safety Algorithms 11, 12, 13 or 14;

FIG. 4 pictorially illustrates one form of a system constructed in accordance with a present invention for guiding the movements of a device by ultrasound, in this case a medical needle, towards a target employing a safety system based on redundant transmitters, where the relative positional relationship between the transmitters is unknown;

FIG. 5a shows the guidance system and safety system of FIG. 1, where the locations of the transmitters and receivers have been switched;

FIG. 5b shows the guidance system and safety system of FIG. 1, where the transmitters are replaced by transcievers and the receivers are replaced by reflectors;

FIG. 6 pictorially illustrates one form of a system constructed in accordance with the present invention for guiding the movements of a device by ultrasound, in this case a medical needle, towards a target, employing a safety system based on redundant receivers;

FIG. 7 diagrammatically illustrates the various vectors involved in using the system of FIG. 6 for guiding the movements of the needle towards the target;

FIG. 8a is a flowchart illustrating Safety Algorithm 21 for the operation of the systems of the present invention;

FIG. 8b is a flowchart illustrating Safety Algorithm 22 for the operation of the systems of the present invention;

FIG. 8c is a flowchart illustrating Safety Algorithm 23 for the operation of the systems of the present invention;

FIGS. 9a-9c illustrate various waveforms associated with a safety system based on signal alternating (hopping);

FIG. 10 pictorially illustrates a second guidance system constructed in accordance with the present invention for guiding the movements of a device by ultrasound, in this case a medical needle, towards a target;

FIG. 11a pictorially illustrates a third guidance system constructed in accordance with the present invention for guiding the movements of a device by ultrasound, in this case a medical needle, towards a target;

FIG. 11b shows the guidance system of FIG. 11a, where the positions of the transmitters and receivers are switched;

FIG. 11c shows the guidance system of FIG. 11a, where the transmitter(s) is/are replaced by a transceiver and the receiver(s) is/are replaced by reflectors;

FIG. 12 diagrammatically illustrates the various vectors involved in using the system of FIGS. 11a-11c;

FIG. 13 pictorially illustrates the third guidance system of the present invention, employing a safety system based on redundant transmitters;

FIG. 14 diagrammatically illustrates the various vectors involved in using the system of FIG. 13 for guiding the movements of the needle towards the target;

FIG. 15 pictorially illustrates the third guidance system of the present invention, employing an alternate safety system based on redundant receivers;

FIG. 16 diagrammatically illustrates the various vectors involved in using the system of FIG. 15 for guiding the movements of the needle towards the target; and;

FIG. 17 pictorially illustrates the third guidance system of the present invention, employing a safety system based on redundant transmitters and redundant receivers.

### DETAILED DESCRIPTION OF THE DRAWINGS

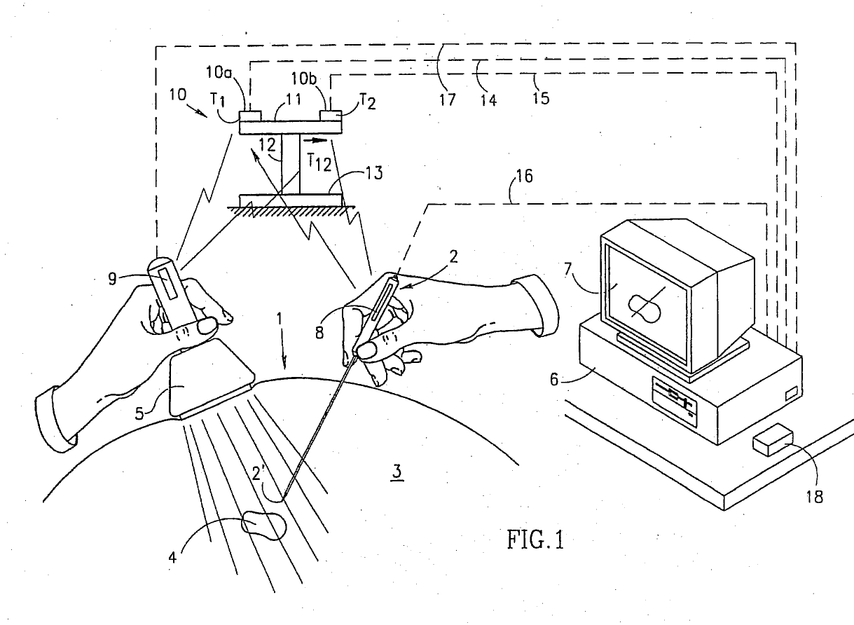

The system illustrated in FIG. 1 shows the invention of the present application embodied in an ultrasound imaging system based on the guidance system described in the above-cited Patent Application PCT/IL96/00050, hereby incorporated by reference (also referred to herein as a first guidance system), in an operating area 1, for guiding a biopsy needle 2 through a body 3 to a target 4 within the body. The body 3, the needle 2, and the target 4, are all imaged by an ultrasound transducer (scanner) 5, or other similar scanning device, connected to a data processor 6 which produces an image of the target 4 on a display screen 7.

A safety system, based on two or more transmitters, at least one of these transmitters being “redundant” or additional, is incorporated with this first guidance system. The needle 2 includes a position sensor 8, typically a receiver (Rₙ), at a predetermined location on the needle 2. The ultrasound transducer 5, also includes a position sensor 9, typically a receiver (Rᵤ), at a predetermined location on it.

Transmitter apparatus, generally designated 10, transmits radiant energy, preferably in signals, such as light, magnetic or electromagnetic radiation at frequencies ranging from approximately DC to approximately high frequency, to the two position sensors 8 and 9. The two position sensors 8 and 9 receive this radiant energy from transmitter apparatus 10, preferably including two transmitters 10a, 10b (T₁, T₂), also known as transmitter units, and produce outputs corresponding to the positions of the needle 2 and the transducer 5, respectively, with respect to a reference location in space occupied by the transmitter apparatus 10. Data processor 6 receives the information from the transmitter apparatus 10, the needle position sensor 8, and the transducer position sensor 9. It processes this data and displays on the display screen 7 the expected position and trajectory of the needle 2 with respect to the target 4, thereby aiding the physician to guide the movement of the needle 2 towards the target 4.

Further details of the construction and operation of the system illustrated in FIG. 1, as well as other similar systems that could be used, are set forth in the above-cited Patent Application PCT/IL96/00050, incorporated herein by reference.

In the system described in PCT/IL96/00050, the transmitter apparatus 10 includes a single transmitter, such as described in U.S. Pat. No. 4,945,305 (Blood), for transmitting the signals to position sensor 8 on the needle 2, and to position sensor 9 on the transducer 5. The position of the transmitter, serves as a reference in space for the two position sensors.

As briefly described above, interfering objects and/or interfering fields, such as electromagnetic fields, within the region, particularly in the vicinity of either of the position sensors 8, 9, may produce measurement errors which if relied upon by the physician in guiding the needle 2, may cause serious injury to the patient. The system illustrated in FIG. 1, therefore, includes an arrangement which is effective to alert the physician, e.g. by the actuation of an alarm 18, in the event that there are interfering objects or interfering fields in the immediate vicinity which could produce measurement errors of a magnitude that could cause injury to the patient, so that the physician will not rely on the displayed measurements until the interfering objects are removed.

For this purpose, the transmitter apparatus 10 preferably includes two transmitters 10a, 10b (T₁ and T₂), also known as transmitter units, one being “redundant” or additional, mounted at the opposite ends of an arm 11 having a known spatial relationship, defined by the vector T₁₂. Arm 11 is supported on a stand 12 mounted on a base 13.

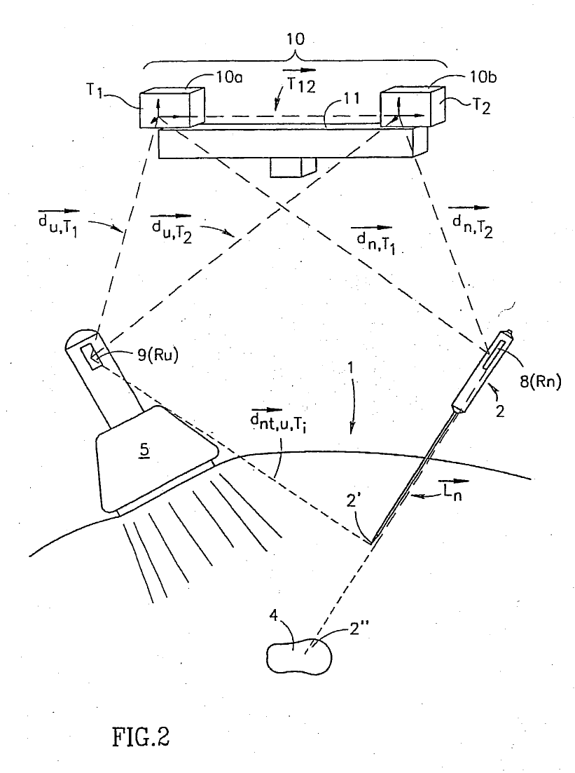

In the example illustrated in the drawings, the two transmitters 10a, 10b (T₁,T₂) are both oriented in arbitrary directions; that is, the two sets of Cartesian coordinates to which the two transmitters are linked may be parallel or non-parallel to each other. As seen in FIG. 2, the separation of the. origins of the coordinates is described by a known and fixed vector \vec{T}₁₂, determined by the construction of the device and therefore known or measurable beforehand. The safety system detailed in FIGS. 1 and 2 is based on transmitter redundancy and the position of each receiver is measured relative to the position of each transmitter unit, the positions of the transmitter units being reference positions.

The magnitude of the arm 11, and thereby of fixed vector \overline{T}₁₂, should be long enough to amplify the effects of the errors, when and if they occur by the presence of an interfering object or interfering fields, but should be short enough to make the device relatively compact. In applications such as described herein, the length of arm 11 is preferably from 0.1 to 1.0 meters, most preferably about 35 cm.

FIG. 2 illustrates the location and orientation of transmitter 10b (T₂) from transmitter 10a (T₁) as represented by the known vector \vec{T}₁₂. The location and orientation of position sensor 8 on the needle 2 is represented by a first vector \vec{d}_{n,t1} with respect to transmitter T₁, and by a second vector \overline{d}_{n,t2} with respect to transmitter T₂. Similarly, the distance and orientation of position sensor 9 on the ultrasound transducer 5 is represented by a third vector \overline{d}_{u,t1} with respect to transmitter T₁, and by a fourth vector \vec{d}_{u,t2}, with respect to transmitter T₂.

The location of the needle tip 2′ relative to the needle position sensor 8 is represented by \vec{L}ₙ, the location of the needle tip relative to the ultrasound position sensor 9 is represented by \vec{d}_{nt,}_{u,Ti}, i=1,2, where the index Tᵢ indicates that the measurement was made according to transmitter T₁, or T₂.

Since the arm 11 carrying the two transmitters 10a, 10b (T₁, T₂) at its opposite ends is fixed in space, the vector \vec{T}₁₂ representing the location and orientation of transmitter T₂ with respect to transmitter T₁, (which as described above is known beforehand) remains fixed during the operation of the system. However, as needle 2 is manipulated during the operation of the system, the first and second vectors \vec{d}_{n,t1} and \vec{d}_{u,t2} representing the location and orientation of the needle sensor 8 with respect to the two transmitters T₁ and T₂, respectively, continuously change with the movement of the needle. Similarly, the third and fourth vectors \vec{d}_{u,t1} and \vec{d}_{u,t2}, representing the location and orientation of the ultrasound transducer position sensor 9 with respect to transmitters 10a and 10b (T₁ and T₂) respectively, also continuously change with the movement of the ultrasound transducer 5.

The output of the position sensors 8, 9 and data for the transmitter apparatus 10 are communicated in any suitable manner, e.g. by wired or wireless links 14, 15, 16, 17 respectively, to the data processor 6. Based on the data received therefrom, the data processor 6 measures the position of the sensors 8, 9 with respect to the transmitters 10a, 10b (T₁ and T₂).

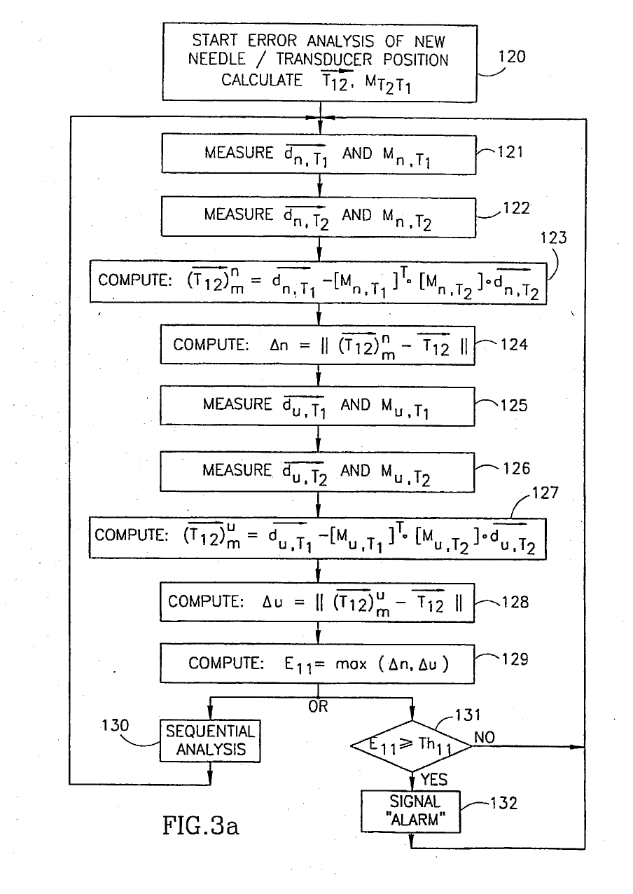

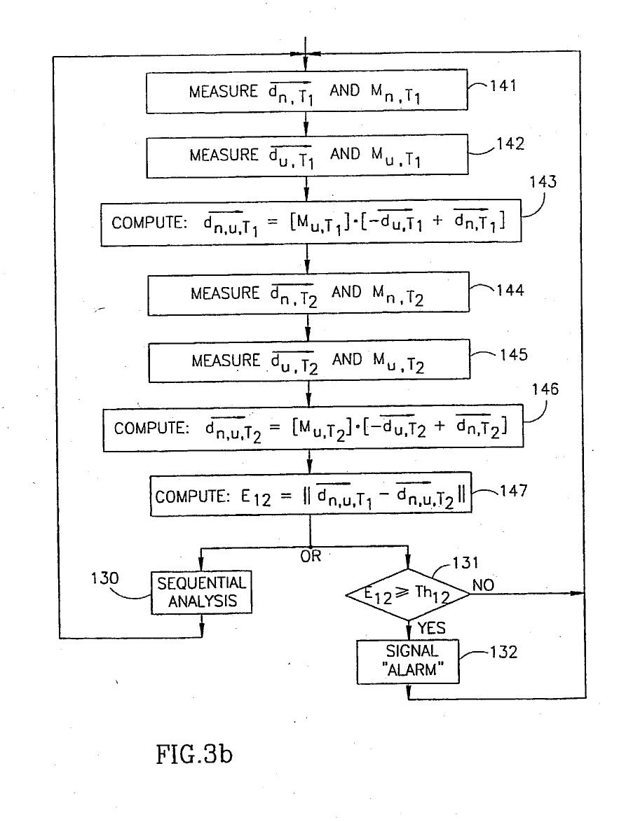

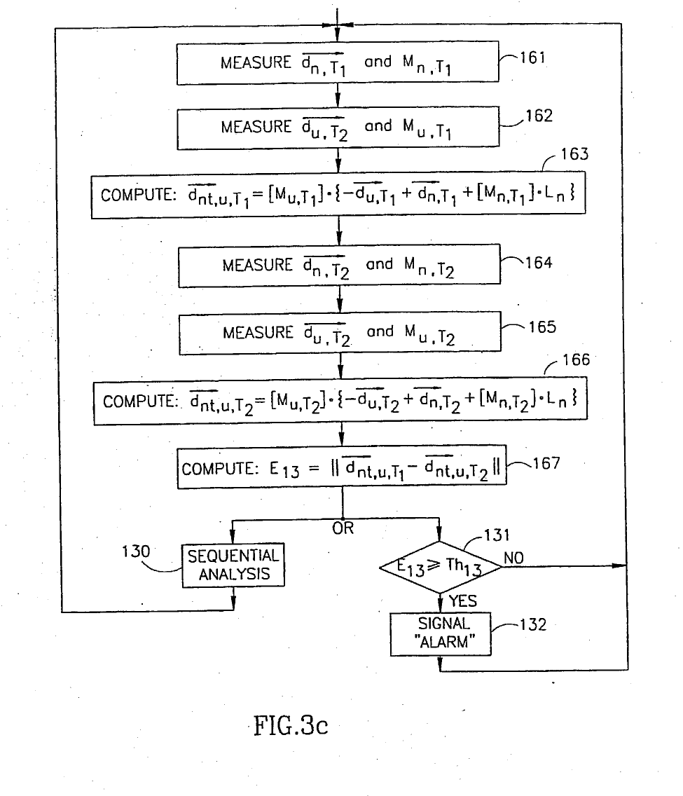

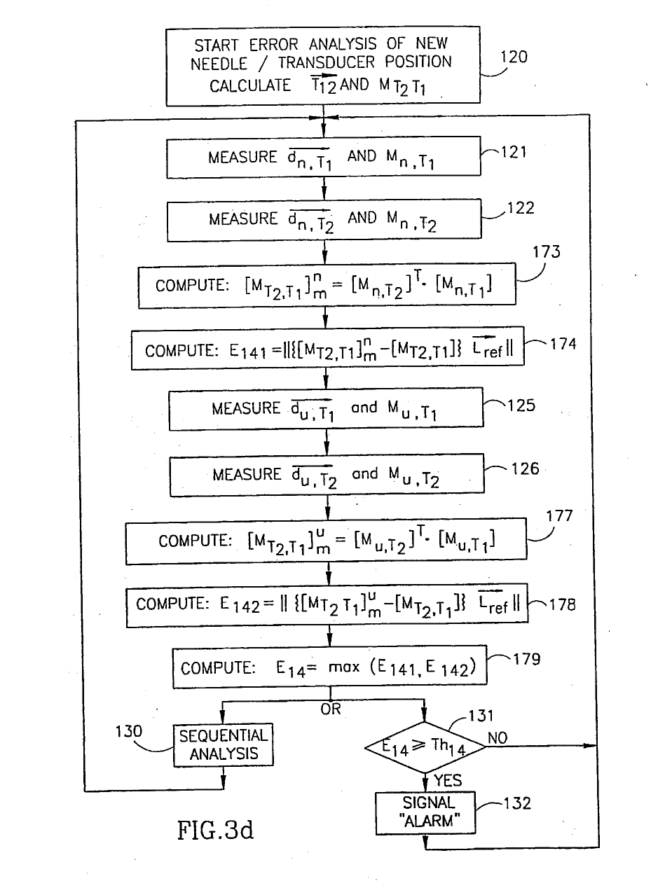

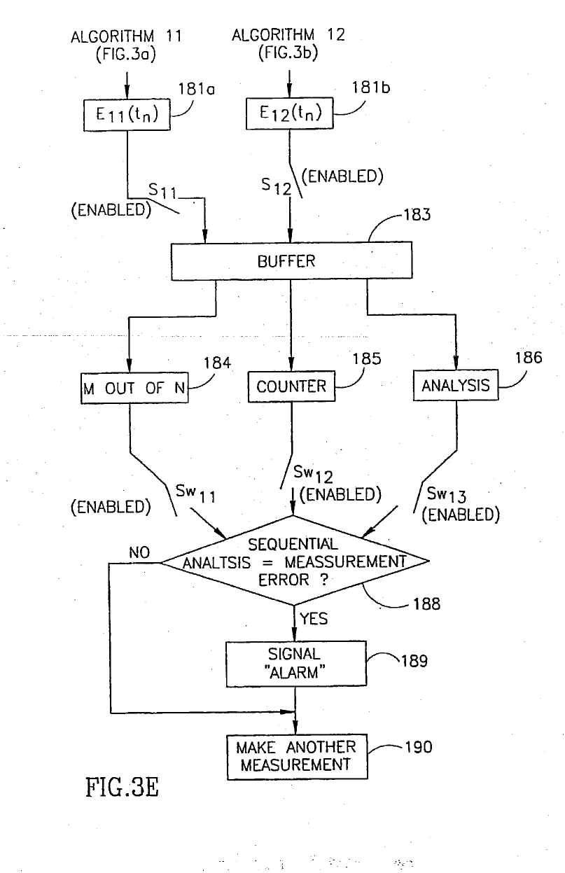

The data processor 6 processes the inputted data to calculate, and display the expected trajectory of the needle 2 towards the target 4 on the display screen 7, in the manner described in the above-cited Application PCT/IL96/00050. However, data processor continuously performs additional operations, preferably in the form of Algorithms 11, 12, 13 and 14, detailed immediately below, and illustrated in FIGS. 3a-3c, constituting an error-analysis procedure.

As described below, the purpose of the error-analysis procedure (as well as that of the safety system) is to alert the physician, as by actuating an alarm 18 (FIG. 1), should there be in the immediate vicinity interfering objects which might produce measurement errors which could lead to injury to the patient should the physician rely on these calculations and on the display on screen 7 for guiding the needle to the target.

Algorithm 11

The vector \overline{T}₁₂ can be determined by a mechanical measurement. It can also be determined by measurement in an interference-free environment. For example, a needle containing a position sensor attached to its tip may be placed in an environment free of interference, and the position of the sensor may then be measured with respect to the coordinates linked to transmitters 10a and 10b (T₁ and T₂). The actual measurement may be by optical means, e.g. lasers, or any other known method for measuring position.

For this algorithm, block 120 illustrated in FIG. 3a (generally measured off-line) is the starting state of the error-analysis procedure performed by data processor 6 to define the vector \overline{T}₁₂. The data processor first measures the location vector distance \vec{d}_{n,t1}; and orientation matrix M_{n,t1} defining the location and orientation of the position sensor 8 on needle 2 with respect to transmitter 10a (T₁) (block 121). It then measures the location vector \vec{d}_{n,t2} and the matrix M_{n,t2}, defining the location and orientation of the needle position sensor 8 with respect to transmitter 10b (T₂) (block 122). From these measurements, the data processor then computes the measured value of the vector (\vec{T}₁₂)ₘ defining the location of transmitter T₂ with respect to transmitter T₁ by making the following computation (block 123):

(\vec{T}₁₂)ₘⁿ=\overline{d}_{n,t1}−[M_{n,t1}]^{T}[M_{n,t2}]\overline{d}_{n,t2} Eq. (1)

The value of the known vector \vec{T}₁₂ between the two transmitters T₁ and T₂ is subtracted from the value of the measured vector between the two transmitters to compute the error (Δn) as follows (block 124):

Δn=∥(\vec{T}_{1,2})ₘⁿ−\vec{T}₁₂∥ Eq. (2)

The steps represented by blocks 121, 122, 123 and 124, are then repeated with respect to position sensor 9 on the ultrasound transducer 5, in blocks 125, 126, 127 and 128, respectively, wherein block 128 computes the error between the known vector \overline{T}₁₂ and the transducer-based measured vector (\vec{T}₁₂)ₘ^{u} by making the following computation:

Δu=∥(\vec{T}_{1,2})ₘ^{u}−\vec{T}₁₂∥ Eq. (3)

In block 129, a computation is made as to the larger of the two errors, determined in blocks 124 and 128, respectively. The larger of the two errors, defined by E₁₁, is then subject to a one step analysis or a sequential analysis in block 130 (detailed in FIG. 3e). The one step analysis consists of comparing E₁₁ with a threshold Th₁₁, (a maximum permissible error) (block 131), which, if exceeded, actuates alarm 18 (block 132). This alerts the physician that the calculations and display on screen 7 are not to be relied upon because of the errors produced by the intervening objects.

Algorithm 12

Implementation of Algorithm 12, as described below and illustrated in FIG. 3b does not require previous knowledge of the position relationship between the two transmitters 10a, 10b (T₁ and T₂).

After measuring the position of the needle sensor 8 and ultrasound transducer sensor 9 with respect to T₁, blocks 141 and 142 (similar to blocks 121 and 125 respectively), the data processors calculates the location of the needle position sensor 8 relative to ultrasound transducer position sensor 9, in block 143 is as follows:

\vec{d}_{n,u,}_{T1}=[M_{u,T1}]?{\vec{d}_{n,}_{T1}−\vec{d}_{u,}_{T1}} (Eq. 4)

Similarly the data processor calculates in block 146 the location of the needle position sensor 8 relative to ultrasound transducer position sensor 9 defined as \overline{d}_{n,u,T2} based on measurements made relative to T₂, as in blocks 144 and 145 (similar to blocks 122 and 126 respectively).

The difference between the computed vectors in block 147, defined as E₁₂ is then calculated as follows:

E₁₂=∥\vec{d}_{n,}_{u,T1}−\vec{d}_{n,}_{u,T2}∥ (Eq. 5)

The value E₁₂ can then be processed in the same manner as E₁₁ in FIG. 3a.

Algorithm 13

The implementation of Algorithm 13, as described below and illustrated in FIG. 3c, does not require knowing the relative spatial relationship between the transmitters 10a, 10b.

Algorithm 13 is similar to Algorithm. 12, except it is based on calculating the position of the needle tip 2′ (referred by subscript “nt”) relative to the ultrasound transducer position sensor 9, this vector expressed as \vec{d}_{nt,u}.

Initially, steps, shown as blocks 161, 162, corresponding to those detailed as blocks 141 and 142 (FIG. 3b above) are performed. The data processor 6 then calculates the position of the needle tip 2′ with respect to the ultrasound transducer position sensor, based on T₁ transmissions, block 163, as follows:

\vec{d}_{nt,u,T1}=[M_{u,T1}]*{\vec{d}_{n,T1}−\vec{d}_{u,T1}+[M_{n,T1}]^{T}*\vec{L}ₙ} (Eq. 6)

Similarly, the data processor calculates in block 166, the position of the needle tip 2′ with respect to the ultrasound transducer position sensor 9, defined as \vec{d}_{nt,u,T2}, based on T₂ transmissions, according to the measurements made relative to T₂ in blocks 164 and 165. The difference between the two vectors, defined as E₁₃ is then calculated in block 167 as follows:

E₁₃=∥\vec{d}_{nt,u,T1}−\vec{d}_{nt,u,T2}∥ (Eq. 7)

The value E₁₃ can then be processed in the same manner as E₁₁ or E₁₂ in FIGS. 3a and 3b above.

A variation to Algorithm 13 can be made by employing in Eq. 6 a vector other than \vec{L}ₙ.

Algorithm 14

Algorithm 14, shown by a flowchart in FIG. 3d, is a variation to Algorithm 11. It begins with the steps detailed as blocks 120-122 as detailed for Algorithm 11 above. The data processor 6 then calculates, based on the measurements made in blocks 121 and 122, the orientation matrix of the transmitter 10b (T₂) with respect to the other transmitter 10a (T₁) in block 173, and defines this orientation matrix as:

[M_{T2,T1}]ₘⁿ

This value for the orientation matrix is then compared with the known orientation matrix, [M_{T2,T1}], in block 174, resulting in output E₁₄₁, as follows:

E₁₄₁=∥{[M_{T2,T1}]ₘⁿ−[M_{T2,T1}]}·\vec{L}_{ref}∥ (Eq.8)

where \vec{L}_{ref} is a predefined vector.

Measurements in accordance with blocks 125 and 126 (detailed above) are then performed, whereby these measurements are used to calculate the orientation matrix, [M_{T2,T1}], in block 177. This value for the orientation matrix is then compared with the known orientation matrix, [M_{T2,T1}], in block 178, resulting in output E₁₄₂ (similar to the calculation made in block 174 above). The maximum for the values E₁₄₁ and E₁₄₂ is calculated in block 179, and defined as E₁₄.

The value E₁₄ can then be processed in the same manner as E₁₁ or E₁₂ in FIGS. 3a and 3b above.

It must be emphasized that in most cases Algorithm 13 and its proposed variations are preferred, since they are based on the same parameters as the guiding calculation (the needle tip 2′ relative to the ultrasound imaging target 4).

A variation to the transmitter redundancy Algorithms 12 and 13 can be based on making the same measurement by N transmitter units, where N>2 and preferably an odd integer. In this case, the data processor 6 checks that more than P (an integer greater than N/2) of the measurements made relative to different transmitter units are in accord in order to clear the measurement.

While Algorithms 11-14 and variations thereof have been disclosed, additional Algorithms for systems having redundant transmitters are also permissible.

If the transmitter units are not transmitting simultaneously, rather one at the time, additional care should be taken in order to avoid effects of movement when applying this method. More specifically, motion should be noticed and/or be compensated for. Such algorithms involving Kalman filters or other linear or non-linear estimators may be employed here.

FIG. 3e is a flowchart illustrating sequential analysis (see block 130 of FIGS. 3a-3d). The input to the sequential analysis can be the output of any of Algorithms 11, 12, 13 or 14, and/or other Algorithms detailed herein (together all separately). The values monitoring measurement errors, such as E₁₁ (block 181a), E₁₂ (block 181b) etc., can be subject to a sequential analysis together or separately as desired by enabling the register switches S₁₁, S₁₂, S₁₃, etc., with the requisite switch S₁₁, S₁₂, enabled. The enabled values are stored in a buffer 183 and then they are subjected to one of the following analysis:

(a) m out of k=checking for m “bad” values out of k measurements (block 184);

(b) Counter=incrementing by Val₁ when receiving a “bad” measurement; and decrementing by Val₂ when receiving a “good” measurement (block 185);

(c) Other Analysis. This involves, for example, sequential analysis in which each safety measurement receives a coefficient (weight) according to its difference from a threshold value (block 186).

The preferred analysis to be employed is chosen by enabling the switch desired, these switches being SW₁₁, SW₁₂ and SW₁₃. The output of the Analysis block is then continuously inputted to a decision-maker block 188, which if necessary signals an alarm (block 189). As long as the guidance system is functioning, the safety system monitors the accuracy of the measurements by continuing to take safety measurements (block 190).

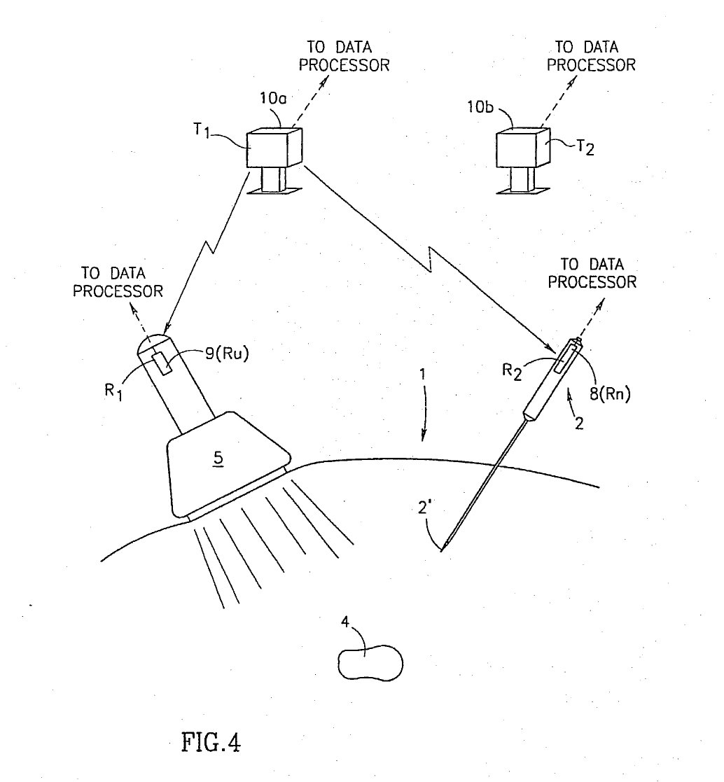

FIG. 4 is similar to the guidance and safety system shown and described in FIG. 1, except the transmitters 10a (T₁) and 10b (T₂) are not necessarily in a known spatial relationship. The vector diagram for the safety system would be in accordance with that shown and described for FIG. 2. Algorithms 12, 13, or variations thereof can be employed with the safety system and method for its use.

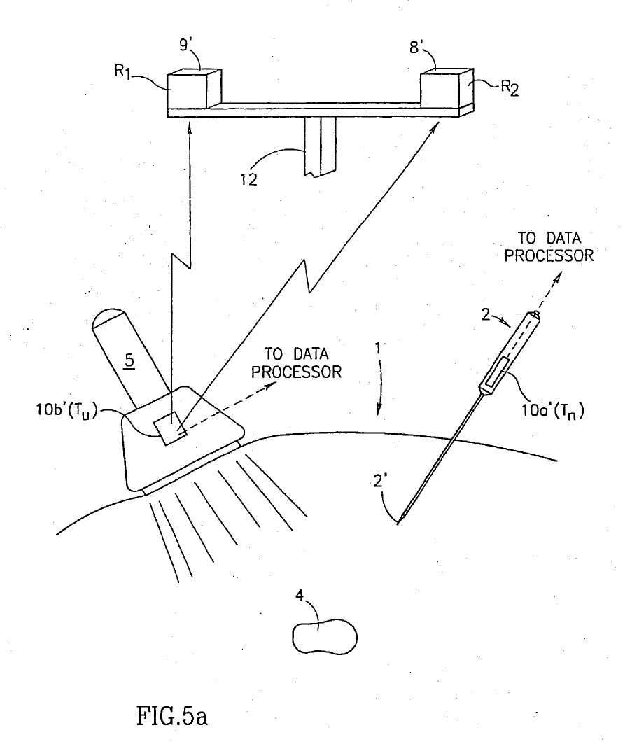

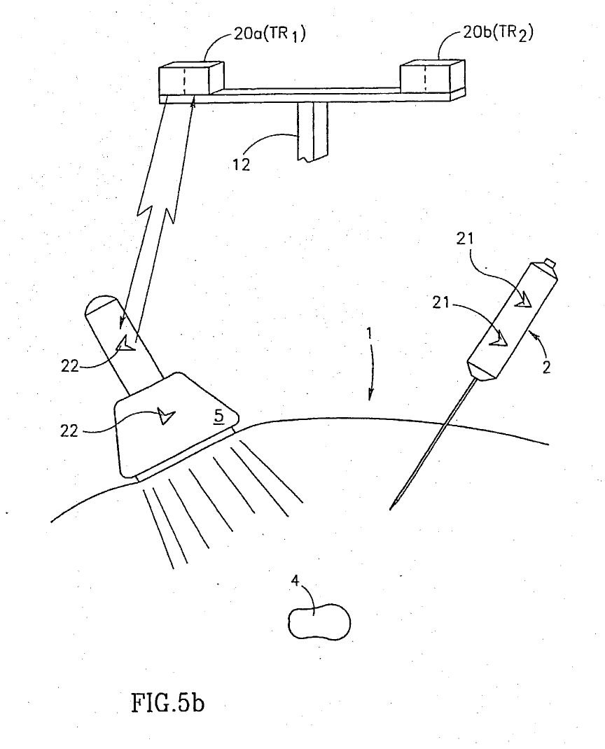

FIG. 5a is similar to the guidance and safety system shown and described in FIG. 1, except the positions of the transmitters T₁ and T₂ and receivers 8 (Rₙ), 9 (Rᵤ) have been switched. The receivers 8′, 9′ (R₁, R₂) (in accordance with receivers 8, 9 detailed in FIG. 1 above) are located on reference positions, and the transmitters 10a′, 10b′ (T^{u} and Tₙ) (in accordance with transmitters 10a and 10b detailed in FIG. 1 above) are affixed to the ultrasound transducer 5 and on the needle 2, respectively. A system comprising only the receiver 9′ (R₁) and transmitters 10a′, 10b′ (T₁, T₂) as illustrated in FIG. 5b, would result in a similar system to that of the first guidance system, as described in PCT/IL96/00050. Measuring the position of the ultrasound transducer 5 and of the needle 2 with respect to the reference location is performed as above. The vector diagram for the guidance and safety system (receivers R₁, R₂ and transmitters Tₙ, Tᵤ) would be in accordance with that shown and described for FIG. 2 (except for replacing the appropriate indexes from Tᵢ to Rᵢ, i=1,2). Algorithms 11, 12, 13, 14 or variations thereof can be employed with the safety system and method, for its use.

FIG. 5b is similar to the guidance and safety system shown and described in FIG. 1, except, the transmitter apparatus is replaced by transceivers 20a, 20b (TR₁, TR₂), typically formed of a transmitter unit coupled with a receiver, and the receivers are replaced by reflectors 21 (RL₁) mounted on the needle 2, and reflectors 22 (RL₂), mounted on the ultrasound transducer 5. The vector diagram for this safety system would be in accordance with that shown and described for FIG. 2. Algorithms 11, 12, 13, 14 or variations thereof can be employed with the safety system and method.

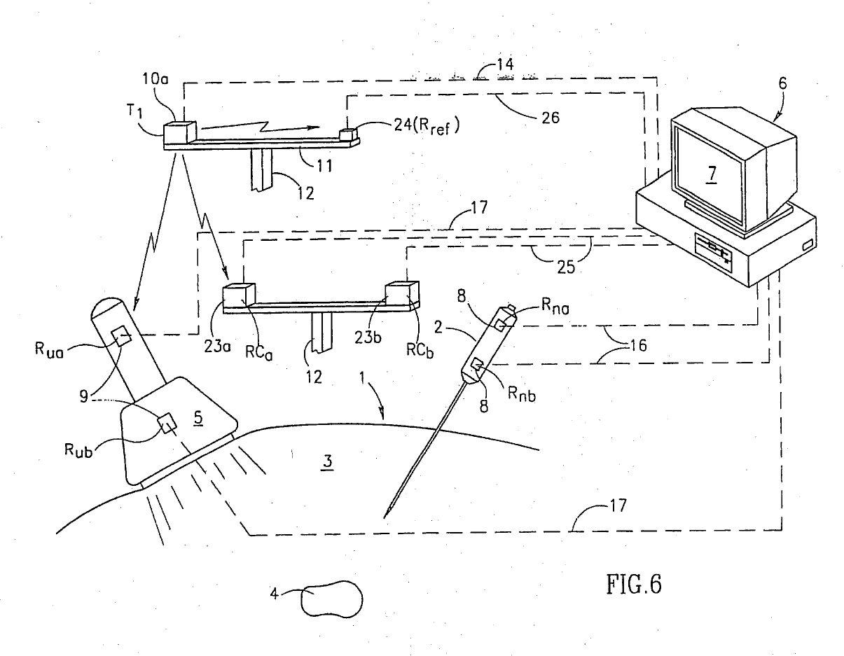

FIG. 6 illustrates a second safety system, based on receiver redundancy, to be employed with the first guidance system as disclosed in our PCT IU/96/00050. The apparatus of the invention would be modified slightly as follows. The transmitter apparatus 10 would include only a single transmitter unit 10a (T₁), although additional transmitter units are also permissible therein. Either the needle 2, the ultrasound transducer 5, or both would have an additional sensor receiver (redundant). It is preferred that the sensors (one sensor being redundant) be spaced as far apart as possible and at different orientations on the device to maximize the probability that any interference will affect each individual sensor 8, 9 (on the needle 2 and ultrasound transducer 5) differently.

Alternatively control position sensor(s) 23a, 23b, typically receivers RCₐ, RC_{b} (in accordance with those detailed above), could be placed in the operating area 1. These control position sensors 23a, 23b would function only to monitor any interference, described above and are placed as a group of sensors with known or fixed positional relationship to each other. Additionally, a reference control position sensor 24, typically a receiver R_{ref} (in accordance with those detailed above) could be placed on a member 11 at a known and fixed position with respect to the transmitter 10a (T₁).

It is to be noted that the safety system based on receiver redundancy to be employed together with said first guiding system can be based on one or more of the above receiver arrangements, and it is not necessary to implement all of the above together.

The sensor(s) 8 (receivers) on the needle 2 and the sensor(s) 9 (receivers) on the ultrasound transducer 5, the transmitter unit 10a (T₁) and the control sensors 23a, 23b (receivers) (if employed) and the reference sensor 24 (receiver) (if employed) are connected by wireless or wired connections 14, 16, 17, 25, 26 (detailed above) to the data processor 6. The data processor 6 then processes the received data, from the sensors 8, 9, 23a, 23b, 24 and transmitter unit 10a (T₁), in accordance with any one of Algorithms 21, 22 or 23 (below), such as to monitor interference and alert the physician in case the measurements are degraded over a predetermined margin.

Alternately, safety systems with both redundant transmitters and redundant receivers are permissible to form the safety system for use with the above detailed first guidance system. Similarly, the above detailed redundant transmitters and redundant receivers could be employed in a safety system with the second and third guidance systems, and alternates thereto, as detailed below.

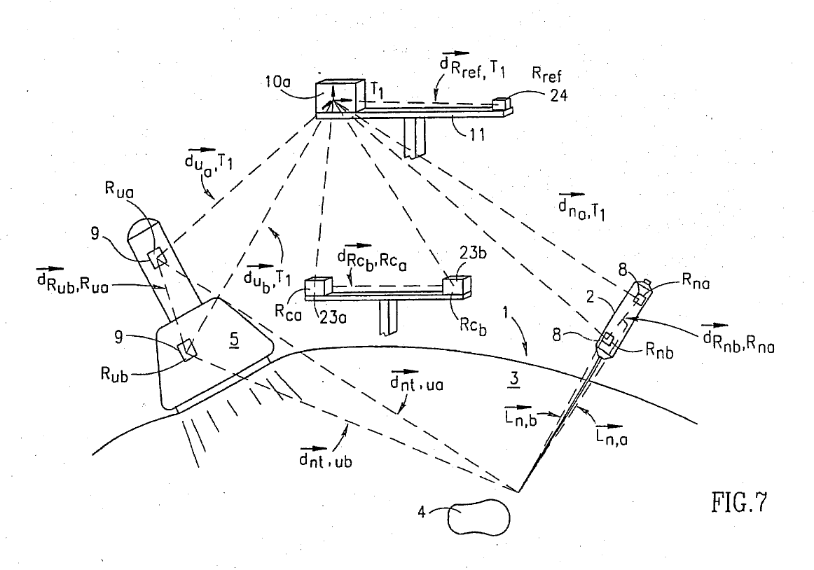

FIG. 7 diagrammatically illustrates the various vectors involved in using the guidance system and safety system detailed in FIG. 6.

Algorithms 21, 22 and 23 as employed with the safety system detailed in FIG. 6 are as follows.

Algorithm 21

Algorithm 21 is based on the assumption that the relative position between two receivers is known. It can be applied to any pair of receivers placed at a known and fixed spatial relationship, such as a pair of sensors 8 (Rₙₐ, R_{nb,}) (receivers) affixed on the needle 2, and/or a pair of sensors 9 (Rᵤₐ, R_{ub,}) (receivers) affixed on the ultrasound transducer 5, and/or a pair of control sensors 23a, 23b (RCₐ, RC_{b}) (receivers), all of these receiver pairs generally referenced below (in the Algorithm) as Rₐ and R_{b}.

The vector between a pair of position sensors can be determined (apriori) by a mechanical measurement. It can also be determined by measurement in an interference-free environment. The actual measurement may be by optical means, e.g. lasers, or any other known method for measuring position.

Algorithm 21 is as follows:

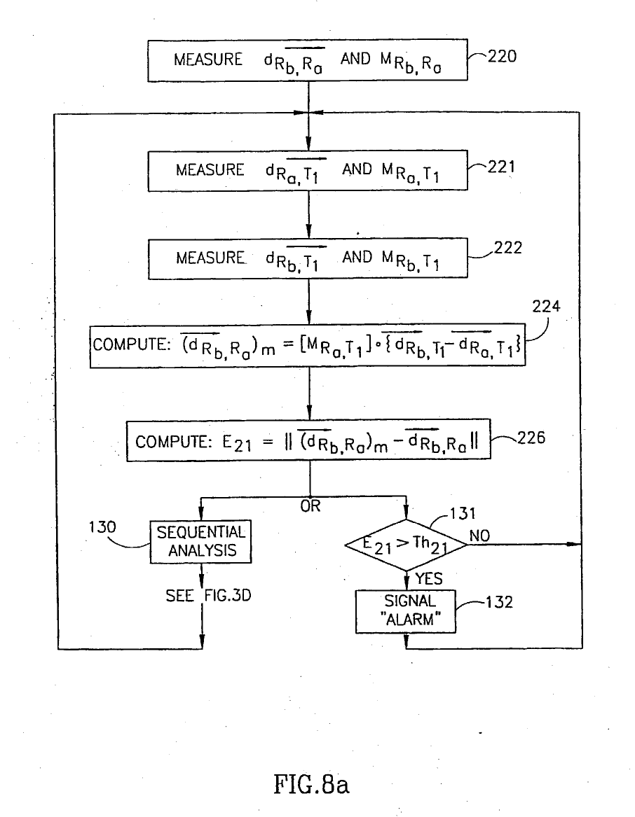

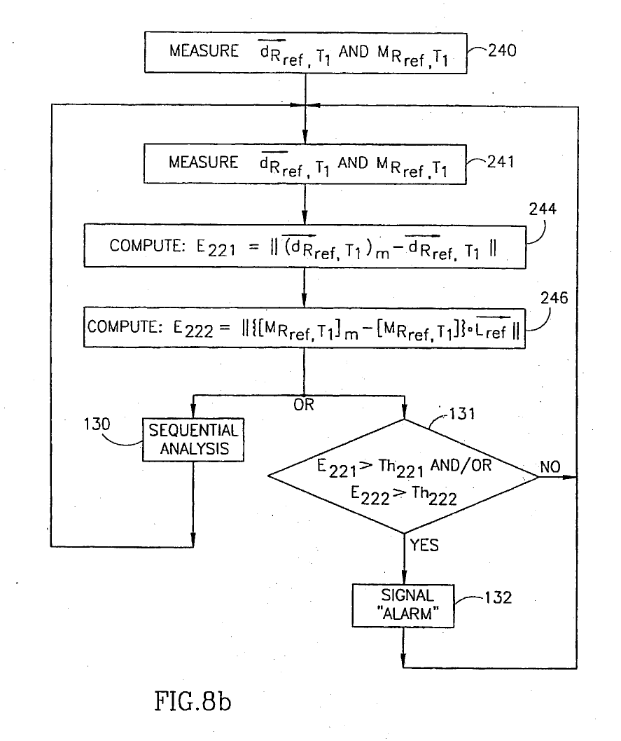

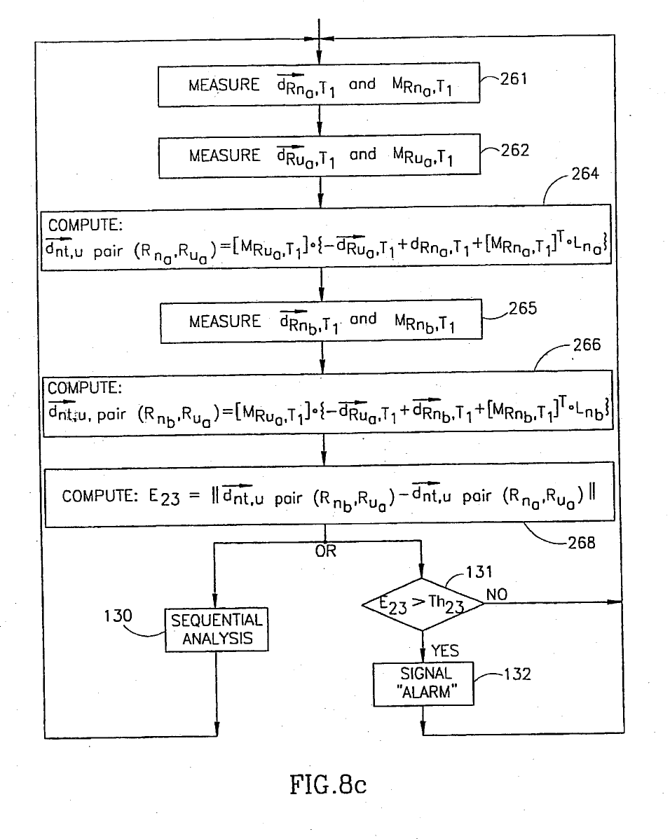

For this algorithm, block 220 illustrated in FIG. 8a (generally measured off-line) to define the vector between the two position sensors is the starting state of the error-analysis procedure.

The data processor measures the position of receiver Rₐ with respect to transmitter unit T₁ (block 221), and defines same as the location vector \vec{d}_{Ra,T1} and orientation matrix M_{Ra,T1}. It then measures the position of receiver R_{b} with respect to transmitter unit T₁, and defines same (block 222) as location vector: \vec{d}_{Rb,T1} and orientation matrix M_{Rb,T1}.

The data processor calculates in block 224 from \vec{d}_{Ra,T1} and M_{Ra,T1} and \vec{d}_{Rb,T1} and M_{Rb,T1} a measured vector (\vec{d}_{Rb,Ra})ₘ between the two receivers: Rₐ and R_{b}. It compares the measured vector (\vec{d}_{Rb,Ra})ₘ with the known vector (\vec{d}_{Rb,Ra}) between the two receivers and produces an output, E₂₁, in block 226, relative to the difference between them.

The value E₂₁ can then be processed in the same manner as E₁₁ in FIG. 3a.

Algorithm 22

Algorithm 22 can be applied whenever a reference control position sensor 24 (R_{ref}) is placed a known and a fixed position with respect with respect to transmitter 10a (T₁). The actual position of the control position sensor R_{ref} with respect to the transmitter T₁, defined as the location vector \vec{d}_{Rref,T1}, and orientation matrix M_{Rref,T1}, can be determined in block 240 similarly to that for the value \vec{T}₁₂ for Algorithm 11 or the value \vec{d}_{Rb,Ra} for Algorithm 21.

The data processor measures the position of receiver R_{ref} with respect to transmitter unit T₁ in block 241, and defines the same as location vector (\vec{d}_{Ref,T1})ₘ and orientation matrix [M_{Ref,T1}]ₘ. The measured value (vector) (\vec{d}_{Ref,T1})ₘ is compared with the known vector \vec{d}_{Ref,T1,} in block 244, to produce an output relative to the difference between them defined as E₂₂₁. The measured orientation matrix [M_{Ref,T1}]ₘ is then compared to the known orientation matrix M_{Ref,T1} in block 246 to produce an output relative to the difference between them, defined as E₂₂₂.

The values E₂₂₁ and E₂₂₂ can then be processed in the same manner as E₁₁ in FIG. 3a.

Algorithm 23

This algorithm can be implemented whenever there are at least two receivers 8 (Rₙₐ and R_{nb})(one of them redundant) affixed to the needle and/or two receivers 9 (Rᵤₐ and R_{ub})(one of them redundant) affixed to the ultrasound transducer 5.

The data processor measures the position of receiver Rₙₐ with respect to transmitter T₁, in block 261, and defines same as location vector \vec{d}_{Rna,T1} and orientation matrix M_{Rna,T1}.

The data processor measures the position of receiver Rᵤₐ with respect to transmitter T₁, in block 262, and defines same as \vec{d}_{Rua,T1} and M_{Rua,T1}.

It then calculates, in block 264, the position of the needle tip 2′, with respect to the ultrasound position sensor 9, based on the measurements of Rₙₐ and Rᵤₐ, and defines same as \vec{d}_{nt,u,pair(R}_{_{na,}}_{R}_{ᵤₐ}₎.

It then measures in block 265 the position of receiver R_{ub} with respect to transmitter 10a (T₁), and defines same as \vec{d}_{Rnb,T1} and M_{Rnb,T1}.

It then calculates, in block 266, the position of the needle tip 2′, with respect to the ultrasound position sensor 9, based on the measurements of R_{nb} and Rᵤₐ and defines same as \vec{d}_{nt,u,pair(R}_{_{nb,}}_{R}_{ᵤₐ}₎.

The data processor compares in block 268 the calculated vectors \vec{d}_{nt,u,pair(R}_{_{na,}}_{R}_{ᵤₐ}₎ and \vec{d}_{nt,u,pair(R}_{_{nb,}}_{R}_{_{ub}}₎, and produces an output E₂₃ as follows:

$${E}_{23} = ‖ {\vec{d}}_{n t , u , pair ( {R}_{na} , {R}_{ua}} - {\vec{d}}_{n t , u , pair ( {R}_{nb} , {R}_{ua}} ‖ \qquad ( Eq . 9 )$$

The value E₂₃ can then be processed in the same manner as the E₁₁ in FIG. 3a.

If an additional redundant receiver (R_{ub}) is fixed on the ultrasound transducer 5, two additional measurements can be added to this Algorithm. These two additional measurements are based on the fact that there are now four pairs of receivers, according to which the needle tip position relative to the ultrasound transducer 5, can be calculated.

A variation to Algorithm 23 is based on making the same measurement relative to N position sensors placed on the said (guided) device, N>2, (preferably odd number). In this case the algorithm checks that more than P (an integer greater than N/2) of the measurements made relative to different position sensors are in accord (in accord defined as within a certain predefined margin) in order to clear the measurement.

While Algorithms 21-23 and variations thereof have been disclosed, additional Algorithms for systems having redundant receivers are also permissible.

The redundant receivers for the safety system above can be employed with the guidance system detailed in FIG. 5a. Here, the receiver(s) serves as the reference position. Further implementation of the system includes the addition of redundant transmitters on the needle 2 and/or the ultrasound transducer 5. This safety system can employ Algorithm 23 as detailed above.

Alternately, redundant transceivers for the safety system above can be employed with the guidance system detailed in FIG. 5b. Here, the transceiver(s) serve as the reference position. Further implementation of the system includes the addition of redundant reflectors on the needle 2 and/or the ultrasound transducer 5. This safety system can employ Algorithm 23 as detailed above.

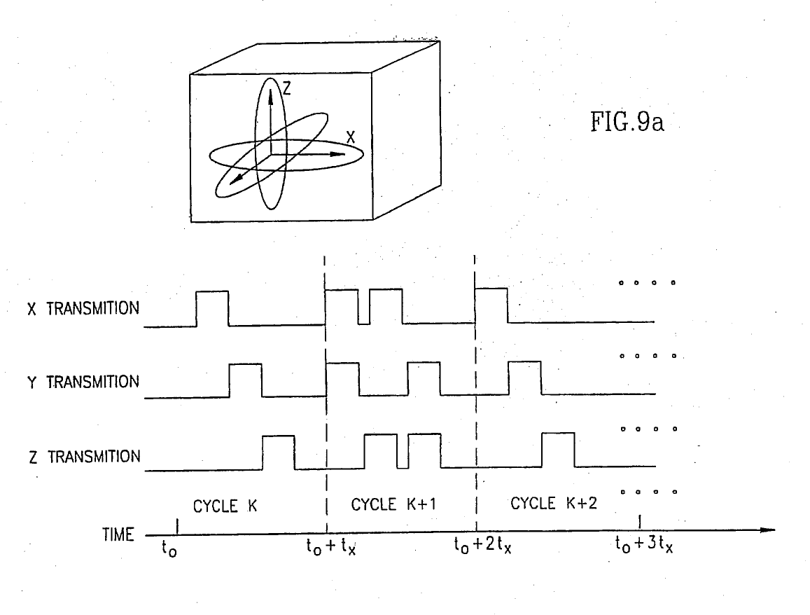

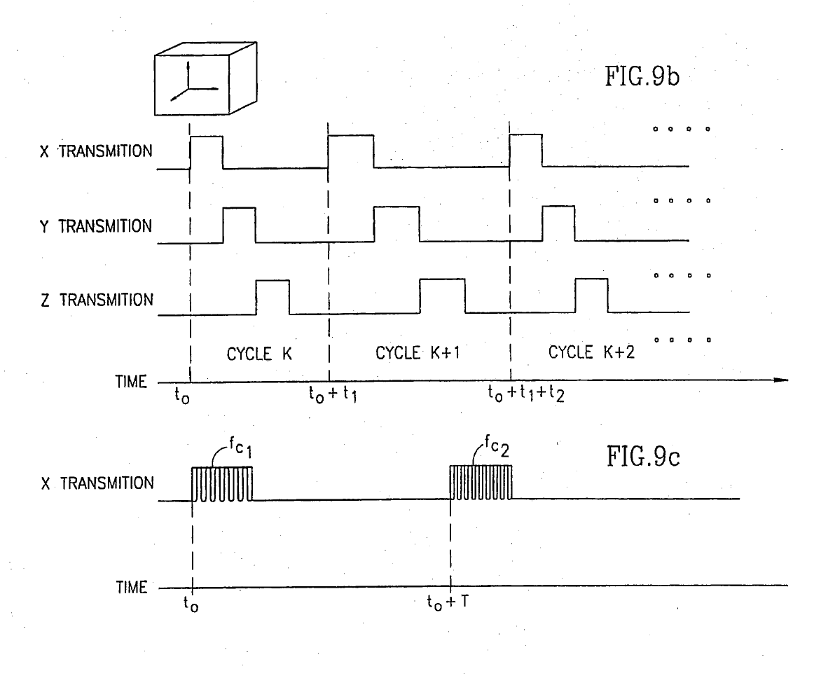

Another safety system, to be employed together with the first guidance system (disclosed in PCT/IL96/00050) (and the guidance systems detailed below), could be transmission/signal/frequency alternating (hopping). For example, in the first guidance system, employing positioning systems as described in U.S. Pat. No. 4,945,305 (Blood) and U.S. Pat. No. 4,054,881 (Raab), both patents incorporated by reference herein, the transmission of transmitter T₁ can be altered, as illustrated in FIG. 9a, and described as follows.

In one cycle of measurements, Cycle K, the transmitter T₁ emits first from antenna X, then from antenna Y, and finally from antenna Z. The following cycle of measurements, Cycle K+1, the transmitter T₁, emits from antennae X and Y (together), then from antennae X and Z (together), and finally from antennae Y and Z (together). The measurements made in the two cycles should be differently affected by interfering objects and/or interfering electromagnetic fields. The data processor 6 measures the needle Up 2′ with respect to the ultrasound position sensor 9 according to measurements made in Cycle K and defines the same as \vec{d_{nt,u,Cycle K}. The data processor then measures the needle tip 2′ with respect to the ultrasound position sensor 9 according to measurements made in Cycle K+1 and defines the same as d}_{nt,u,Cycle K+1}. It then compares \vec{d}_{nt,u,Cycle K} and \vec{d}_{nt,u,Cycle K+1} and then produces an output equal to the difference between the two measurements, defined as Eₛ₁, whose value can then be processed in the same manner as the value E₁₁ in FIG. 3a.

U.S. Pat. No. 4,945,305 (Blood) describes both types of measurement cycles, however as separate implementations of the positioning system and not in a possible combined system in order to monitor measurement errors caused by interference.

Another safety system, based on signal alternation, to be employed together with said first guidance system, when employing a positioning system as described in U.S. Pat. No. 4,945,305 (Blood), is illustrated in FIG. 9b and described below.

The transmitter, as detailed in the '305 (Blood) patent, emits different pulse shapes and/or pulse lengths in consequent measurement cycles, e.g., Cycle K and Cycle K+1. Data processor 6 calculates \vec{d}_{nt,u,Cycle K} and \vec{d}_{nt,u,Cycle K+1} (both defined above). The measurements made in the two cycles should be differently affected by interfering objects and/or interfering electromagnetic fields. The data processor 6 then compares the above measurements and produces an output equal to them, defined as Eₛ₂, whose value can then be processed in the same manner as the E₁₁ in FIG. 3a.

When employing a system as described in U.S. Pat. Nos. 4,054,881 and 4,314,251(both to Raab), this '251 patent is also incorporated by reference herein, a safety system based on frequency hoping can be employed, as illustrated in FIG. 9c and described below.

The transmitter emits on different frequency carriers on consequent measurement cycles (frequency hoping). The measurements made in the two cycles should be differently affected by interfering objects and/or interfering electromagnetic fields. Therefore, the data processor can compare between the measurements and produce an output equal to the difference between the two measurements, defined as Eₛ₃, whose value can then be processed in the same manner as the value E₁₁ in FIG. 3a.

An additional safety system to be employed with said first guidance system, when using positioning systems as described in the '305 (Blood) patent, and the '881 (Raab) and '521 (Raab) patents, can be made by checking the unitarity of matrix A, this matrix A being the receiver allitude matrix in the '305 patent. Accordingly, the data processor calculates:

E_{un,1}=matrix_{—}norm(A^{T}*A−I) (Eq. 10)

Eᵤₙ₂={λₘₐₓ(A^{T}*A)/λₘᵢₙ(A^{T}*A)} (Eq. 11)

where, matrix_norm stands for matrix norm, and λₘₐₓ(A^{T}*A), λₘᵢₙ(A^{T}+*A) stand for maximal eigenvalue and minimal eigenvalue of matrix A^{T}*A.

The values of Eᵤₙ₁ or Eᵤₙ₂ can then be processed in the same manner as the E₁₁ in FIG. 3a.

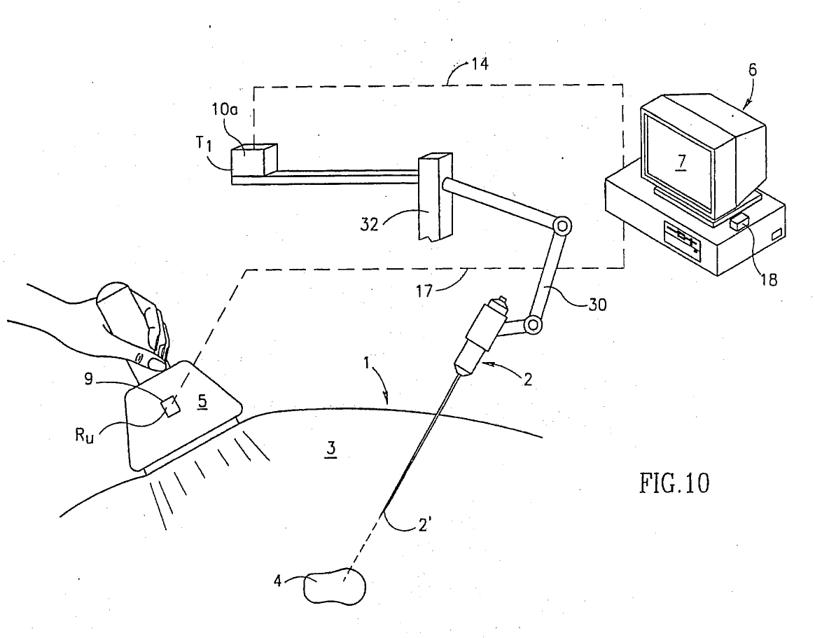

FIG. 10 is directed to a second guidance system. More particularly, it is directed to provide a second guidance system and method that may be used for guiding medical devices towards a target in a body.

In this guidance system, all components remain the same and function similarly to those of the first guidance system detailed in the above cited PCT/IL96/00050, except the needle 2 is mounted on an articulated arm 30, mounted on a stand 32. The articulated arm 30 and stand 32 are disclosed in U.S. Pat. No. 5,647,373, assigned to the assignee of the present invention, the disclosure of which is incorporated by reference in its entirety herein.

A transmitter 10a (T₁) is positioned at a reference position on the stand 32. A sensor 9, preferably a receiver Rᵤ, is placed on the ultrasound transducer 5. Both the sensor 9, and the transmitter 10a communicate with the data processor 6 by wired or wireless links 14, 17, detailed above, enabling the data processor 6 to measure the position of the ultrasound transducer 5 with respect to the reference position as described above.

This second guidance system and alternates, detailed above, can have its receivers and transmitters arranged such that a positioning and tracking system is defined. These receivers and transmitters could be modified such that the desire positioning and tracking system are magnetic, acoustic, or electro-optical or combination thereof (in addition to that detailed above), in accordance with PCT/IL96/00050.

The arm 30 is mechanically calibrated to the reference position, such that its movements, including those of the needle 2, are sent to the data processor 6, by wired or wireless connections as detailed above. This enables the data processor 6 to measure the position of the needle with respect to the reference position. The data processor 6 calculates from these measurements, the position of the needle 2 with respect to the ultrasound imaging plane. The expected trajectory of the needle 2 with respect to the target 4 is now viewable on the display screen 7, thereby aiding the physician to guide the movement of the needle 2 toward the target 4.

This second guidance system and alternates may also be employed together with other scanning apparatus such as computerized tomography, X ray, as detailed in the above cited PCT/IL96/00050.

All the above safety systems, as described in connection with the first guidance system, can be employed with this second guidance system. For example, a safety system based on redundant transmitters is formed when a second transmitter (T₂) is placed on the stand 32. These “transmitter redundant” safety systems would have vector diagrams in accordance with that detailed in FIG. 2 above, and could employ any of the safety algorithms, detailed as Algorithms 11, 12, 13 and 14 above.

Alternately, two or more, position sensors, receivers, (at least one being redundant) could be placed on the ultrasound transducer 5, or control position sensors (23a, 23b and 24 as shown in FIG. 6) could be placed in the operating area 1, as described above for FIG. 6, resulting in receiver redundant safety systems, in accordance with those described above. These “receiver redundant” safety systems would have vector diagrams in accordance with that detailed in FIG. 7 above, and could employ any of the safety Algorithms, detailed as Algorithms 21, 22 and 23 above.

Safety systems based on signal alternating as described above can also be employed together with these second types of guidance systems.

Additional variations to said second type of guidance systems could be made by exchanging the position of the transmitter T₁ and receiver Rᵤ, and placing the receiver at the reference position, or replacing the transmitter by a transceiver and the receivers by reflectors, as described above.

An alternate guidance system based on that shown in FIG. 10, is formed when the needle 2 is free and the ultrasound transducer 5 is mounted on the articulated arm 30 and stand 32, as detailed above. The needle 2 includes a sensor 8, preferably a receiver (R₁), attached thereto, and the stand 32 includes a transmitter (T₁), attached thereto, as detailed above, both the sensor 8 and Transmitter (T₁) communicating with the data processor 6 by wired or wireless links, detailed above. All above variations to the second guidance system apply to s this type of system also.

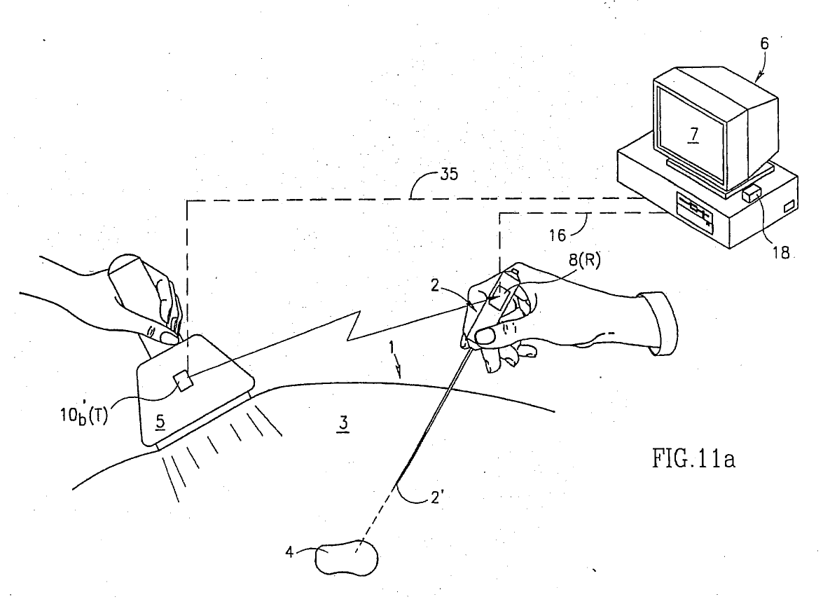

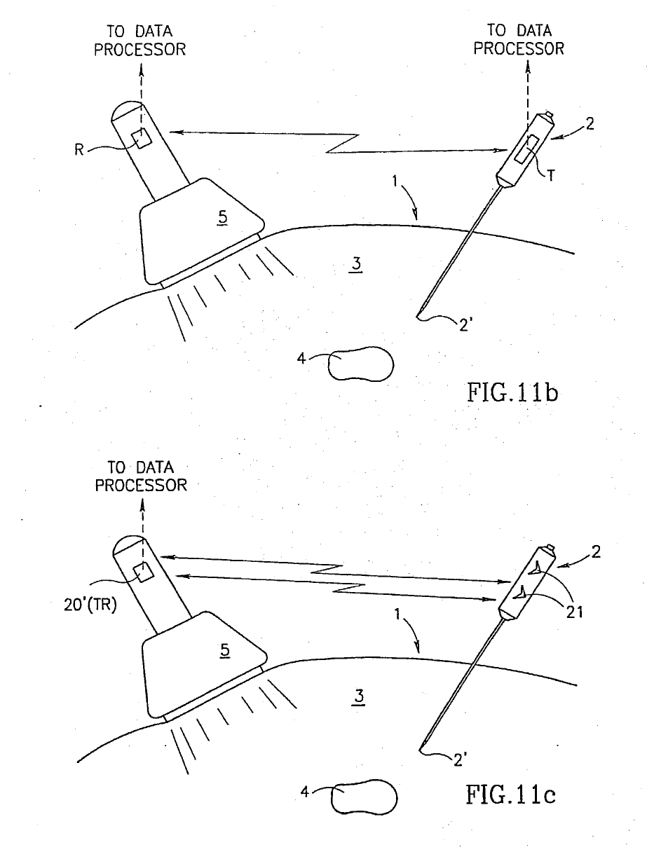

FIG. 11a is directed to a third guidance system. More particularly, it is directed to provide a third guidance system and method that may be used for guiding medical devices towards a target in a body.

This third guidance system differs from the first and second guidance systems detailed above, in that it directly measures the position of the needle 2 with respect to the ultrasound transducer 5 without employing an additional reference location in space. (In the first and second guidance systems, the position of the needle 2 and the position of the ultrasound transducer 5 are first measured, relative to a reference position in space. From these two measurements, the relative position of the needle 2 with respect to the ultrasound transducer 5 is calculated, as detailed above and in PCT/IL96/00050.)

This third guidance system includes a position sensor 8, typically a receiver (R) attached to the needle 2, as detailed above and a transmitter 10b′ (T) (in accordance with transmitter 10b detailed in FIG. 1 above), affixed to the ultrasound transducer 5. The position sensor 8 and the transmitter 10b′ (T) communicate with the data processor 6 by wired or wireless links 16, 35, as detailed above. The position of the needle tip 2′ with respect to the transmitter 10b′ (and therefore to the scanning device and scanning plane) is then measured directly, without the need of an additional reference position (as for said first guidance system), according to the following equation:

\vec{d}_{nt,u}=\vec{d}_{n,u+[}M_{n,u}]^{T}·\vec{L}ₙ (Eq. 12)

The expected trajectory of the needle 2 with respect to the target 4 is now viewable on the display screen 7, thereby aiding the physician to guide the movement of the needle 2 toward the target 4 as described in PCT/IL96/00050.

This third guidance system and alternates may also be employed together with other scanning apparatus such as computerized tomography, X-ray, as detailed in the above cited PCT/IL96/00050.

This third guidance system and alternates, detailed above, can have its receivers and transmitters arranged such that a positioning and tracking system is defined. These receivers and transmitters could be modified such that the desire positioning and tracking system are magnetic, acoustic, or electro-optical or combination thereof (in addition to that detailed above) in accordance with PCT/IL96/00050.

FIG. 11b shows an alternate system to that shown in FIG. 11a. In this system, the positions of the receiver R and transmitter T have been switched on the needle 2 and ultrasound transducer 5 respectively.

FIG. 11c shows an alternate system to that shown in FIG. 11a. In this system, the transmitter 12 has been replaced by a transceiver 20′ (detailed above), and the receiver has been replaced by reflectors 21. Alternately, the transceiver is positioned on the needle 2 and the reflectors 21 are positioned on the ultrasound transducer 5.

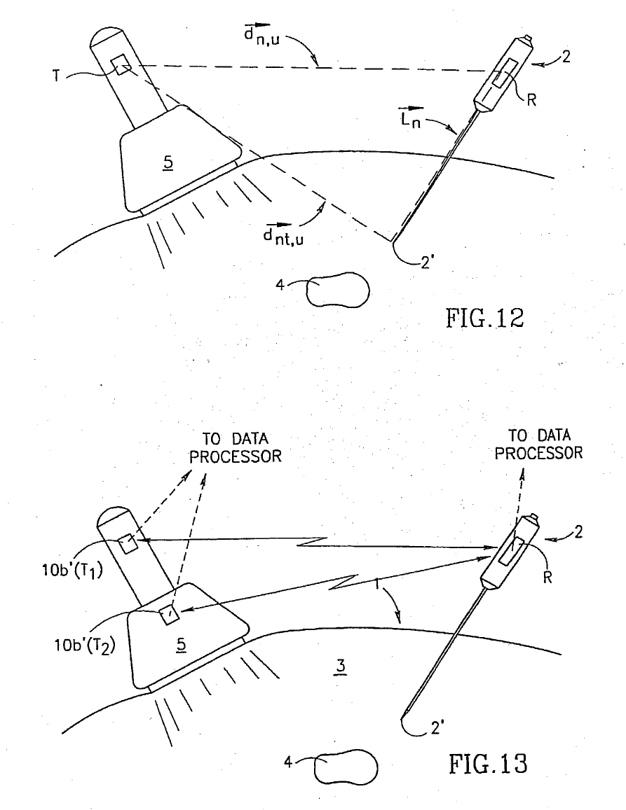

FIG. 12 shows a vector diagram guidance calculation performed for the system of FIG. 11a, that could be modified in accordance with the principles therewith, so as to be applicable to FIGS. 11b and 11c and alternates thereto.

All of the above detailed safety systems can be employed with this third guidance system and alternates shown and detailed above. For example, FIG. 13 shows a safety system based on redundant transmitters. This safety system is used in conjunction with the third guidance system as a sensor 8, typically a receiver R, is on the needle 2 and at least two, preferably two, transmitters 10b′ (T₁, T₂) are on the ultrasound transducer 5, such that one of the transmitters is “redundant” (as detailed above). All transmitters and receivers would communicate with the data processor 6 by wired or wireless communications, as detailed above. Alternately, the transmitters (T₁, T₂) could be on the needle 2 and the sensor (receiver) 8 on the ultrasound transducer 5. These transmitter redundant safety systems could employ any of the safety algorithms detailed as Algorithms 11, 12, 13 or 14 above, with the necessary adjustments to account for the changes in vectors.

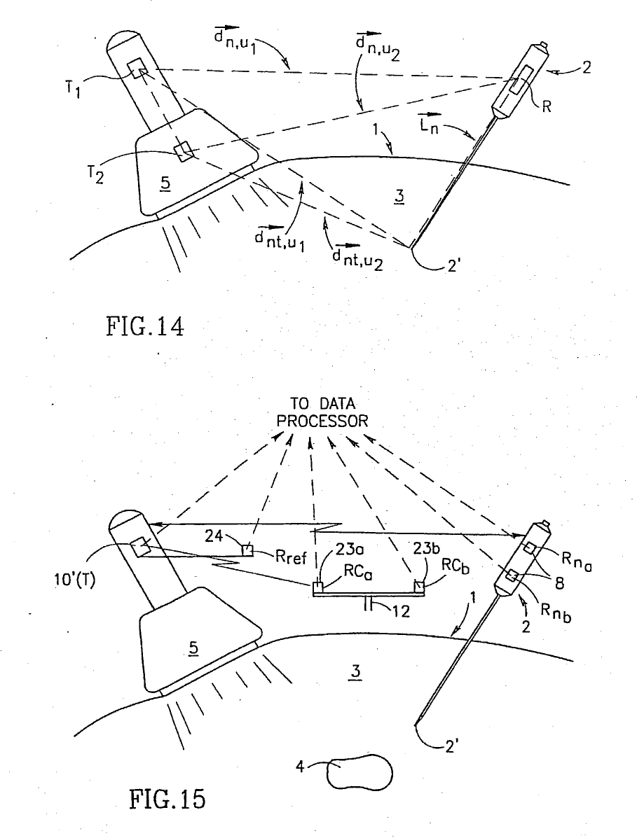

FIG. 14 shows a vector diagram for the system of FIG. 13. This diagram could be modified in accordance with the principles therewith, so as to be also applicable to the alternates thereto.

FIG. 15 details a safety system employing redundant receivers. It is possible to attach two position sensors 8 (receivers) (Rₙₐ, R_{nb}) (at least one of these receivers being redundant), on the needle 2. A transmitter 10b′ (T) is attached to the ultrasound transducer 5. Alternately, control receivers 23a, 23b (in accordance with those above) (RCₐ, RC_{b}) at a known spatial relationship can be placed in the operating area 1. Additionally, a reference position sensor (control receiver) 24 (R_{ref}) (in accordance with that detailed in FIG. 6 above) is preferably placed at a fixed and known position, from the transmitter (as described in relation to first guidance system). Alternately, the two receivers 8 could be on the ultrasound transducer 5, while the transmitter 10b′ could be on the needle 2. All transmitters and receivers would communicate with the data processor 6 by wired or wireless communications, as detailed above. These receiver redundant safety systems could employ any of the safety algorithms detailed as Algorithms 21, 22 or 23 above, with the necessary modifications that account for the changes in vectors.

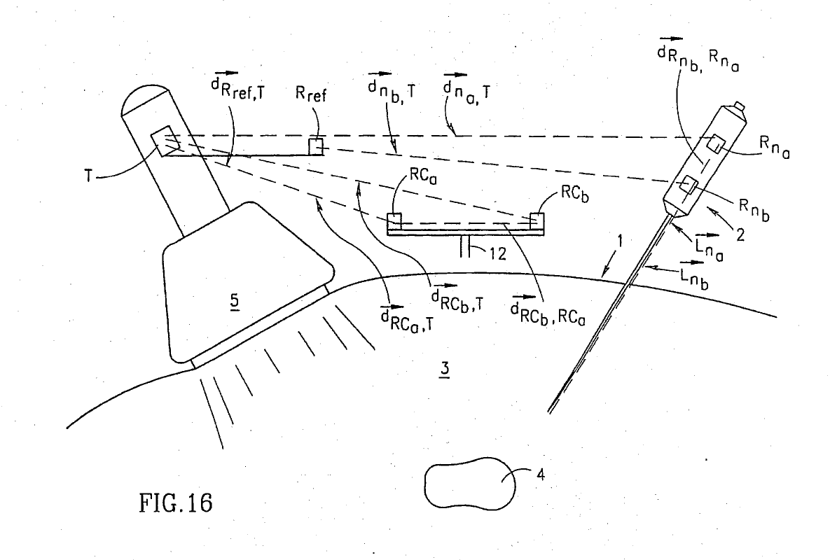

FIG. 16 shows a vector diagram for the receiver redundant system detailed in FIG. 15 above.

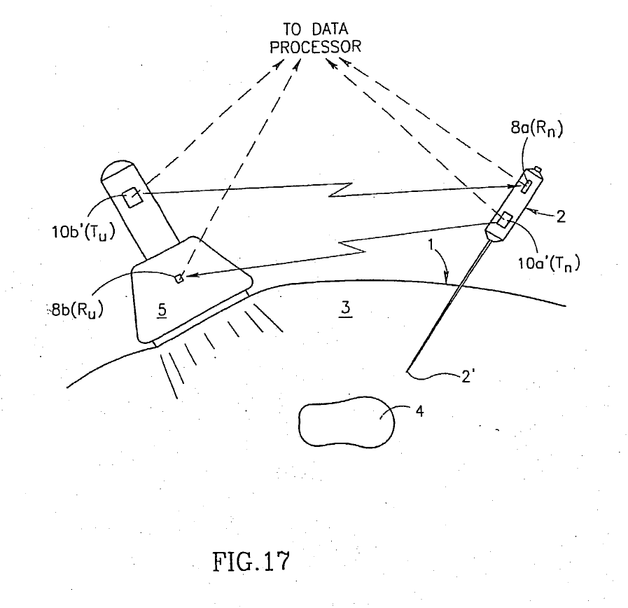

FIG. 17 details a safety system employed with the third guidance system that is both transmitter and receiver redundant. A position sensor 8, typically a receiver (Rₙ) as detailed above, and a transmitter 10a′ (Tₙ) (in accordance with transmitter 10a detailed in FIG. 1 above) are on the needle 2; and a position sensor 9, typically a receiver (Rᵤ), as detailed above, and a transmitter 10b′ (Tᵤ) are on the ultrasound transducer 5. All transmitters and receivers would communicate with the data processor 6 by wired or wireless communications, as detailed above.

Alternately, safety systems with both redundant transmitters and redundant receivers are permissible to form the safety system for use with the above detailed second and third guidance systems.

Safety systems based on signal alternating as described above can also be employed together with these third types of guidance systems.

While the invention has been described with respect to several embodiments, it will be appreciated that this is set forth merely for purposes of example, and that many variations, modifications and other applications of the invention may be made. Rather, the scope of the invention is defined by the claims that follow.

1. A system for guiding the movements of a device towards a target, comprising:
transmitter apparatus at a reference location in space for transmitting radiant energy from said reference location;
a position sensor on said device to be guided to the target, said position sensor receiving radiant energy from the transmitter apparatus and producing an output corresponding to the position of said device with respect to said reference location in space;
and a data processor receiving the output of said position sensor and calculating the position of said device with respect to said reference location:
characterized in that said transmitter apparatus includes a first transmitter (T1), and a second transmitter (T2) in a known spatial relationship to each other defined by a known vector (\vec{T}₁₂)
and further characterized in that said data processor:
(a) produces a measurement of the location and orientation of the position sensor with respect to said first transmitter (T1) and defines same by a first vector;
(b) produces a measurement of the location and orientation of the position sensor with respect to said second transmitter (T2) and defines same by a second vector;
(c) calculates from the said first vector and said second vector a measured vector ( [symbol] ₁₂)m between the two transmitters;
(d) compares the measured vector ( [symbol] ₁₂)m with the known vector (\overline{T}₁₂); and
(e) produces an output corresponding to the difference between the measured vector (\overline{T}₁₂)m and the known vector (\overline{T}₁₂).

2. The system according to claim 1, wherein said data processor includes an alarm, compares said output produced in step (e) with a predetermined threshold value, and actuates said alarm if the output produced in step (e) exceeds said predetermined threshold value.

3. The system according to either of claims 1 or 2, wherein said data processor compares the absolute values of said measured vector (\vec{T}₁₂)ₘ and of the known vector (\vec{T}₁₂).

4. The system according to either of claims 2 or 3, wherein said device is a medical device to be guided through biological tissue in a body to a target therein.

5. The system according to claim 4, wherein said medical device is an interventional device, and said position sensor is located at a predetermined location with respect to said interventional device.

6. The system according to claim 5, wherein said body is imaged by an imaging system which scans the body by a scanning device along a plurality of scan planes.

7. The system according to claim 6:
wherein said scanning device also includes a second position sensor receiving radiant energy from said first and second transmitters and producing an output corresponding to the position of said second position sensor, and thereby of said scanning device, with respect to said reference location in space;
and wherein said data processor performs said steps (a)-(e) also with respect to said second position sensor to produce an output error with respect to said measured vector (T₁₂)ₘ^{u} for the scanner, and utilizes, the larger of said two output errors. for comparison with said predetermined threshold and for actuating said alarm.

8. The system according to claim 7, wherein said scanning device is an ultrasound scanner.

9. The system according to any one of claims 1-8, wherein said first and second transmitters are mounted at the opposite ends of an arm having a known length and orientation in space with respect to said known location in space defining said known vector (\vec{T}₁₂).

10. The system according to claim 9, wherein said arm has a length of 0.1 to 1.0 meters.

11. The system according to claim 10, wherein said arm has a length of 35 cm.

12. A method for guiding the movements of a device towards a target, comprising:
transmitting radiant energy from transmitter apparatus at a reference location in space;
providing a position sensor on said device to be guided to the target, said position sensor receiving radiant energy from the transmitter apparatus and producing an output corresponding to the position of said device with respect to said reference location in space;
processing in a data processor the output of said position sensor and calculating the position of said device with respect to said reference location:
characterized in that said transmitter apparatus includes a first transmitter (T₁), and a second transmitter (T₂), in a known spatial relationship to each other defined by a known vector (\vec{T}₁₂);
and further characterized in that said data processor:
(a) produces a measurement of the location and orientation of the position sensor with respect to said first transmitter (T₁) and defines same by a first vector;
(b) produces a measurement of the location and orientation of the position sensor with respect to said second transmitter (T₂) and defines same by a second vector;
(c) calculates from said first vector and from said second vector a measured \vec{vect}or (T₁₂)ₘ between the two transmitters;
(d) compares the measured vector (\vec{T}₁₂)ₘ with the known \vec{ } (T₁₂) vector; and
(e) produces an output corresponding to the difference between the measured vector (\vec{T}₁₂)ₘ and the known vector (\vec{T}₁₂).

13. The method according to claim 12, wherein said data processor compares said output produced in step (e) with a predetermined threshold value, and actuates an alarm if the output produced in step (e) exceeds said predetermined threshold.

14. The method according to either of claims 12 or 13, wherein said data processor compares the absolute values of said measured \vec{ } vector (T₁₂)ₘ and of the kn\vec{own} vector (T₁₂).

15. The method according to any one of claim 12-14, wherein said known vector (\overline{T}₁₂) is mechanically measured.

16. The method according to any one of claim 12-14, wherein said known vector (\overline{T}₁₂)ₘ is determined by attaching a position sensor to the tip of a needle, placing the needle in an environment free of interference, and measuring the position of said latter position sensor with respect to said first and second transmitters.

17. The methods according to any one of claims 12-16, wherein said device is a medical device to be guided through biological tissue in a body to a target therein.

18. The method according to claim 17, wherein said medical device is a biopsy needle, and said position sensor is located at a predetermined location with respect to said biopsy needle.

19. The method according to claim 18, wherein said body is imaged by an imaging system that scans the body by a scanning device along a plurality of scan planes.

20. The method according to claim 19, wherein said scanning device also includes a second position sensor receiving radiant energy from said first and second transmitters and producing an output corresponding to the position of said second position sensor, and thereby of said scanning device, with respect to said reference location in space;
and wherein said data processor performs said steps (a)-(e) also with respect to said second position sensor to produce an output error with respect to said measured vector (\overline{T}₁₂)ₘ^{u} for the scanner, and utilizes, the larger of said two output errors for comparison with said predetermined threshold and for activating said alarm.

21. The method according to claim 20, wherein said scanning device is an ultra-sound scanner.

22. The method according to any one of claims 12-14, wherein said first and second transmitters are mounted at the opposite ends of an arm having a known length and orientation in space with respect to said reference location in space defining said known vector (\vec{T}₁₂).

23. The method according to claim 22, wherein said arm has a length of 0.1 to 1.0 meters.

24. The method according to claim 23, wherein said arm has a length of 35 cm.

25. A system for guiding the movements of a device toward a target viewable from an image, comprising:
at least one scanning apparatus for creating said image including said target;
at least one transmitter at a first reference location for transmitting radiant energy from said reference location;
at least one first position sensor on said device for receiving said radiant energy;
at least one second position sensor on said at least one scanning apparatus for receiving said radiant energy;
at least one third position sensor for receiving said radiant energy on either said device or said at least one scanning apparatus; and
a data processor in operative communication with said at least one transmitter and each of said at least one first, second and third said position sensors, for monitoring the accuracy of the calculated position of said device with respect to said at least one scanning apparatus.

26. The system of claim 25, wherein said each of said at least one first, second and third position sensors includes a receiver.

27. The system of claim 25, wherein said each of said at least one position sensors is one position sensor.

28. The system of claim 25, wherein said first reference location is a known and fixed location in space.

29. The system of claim 25, wherein said reference location is unknown.

30. The system of claim 25, wherein said at least one scanning apparatus includes one scanning apparatus.

31. The system of claim 30, wherein said at least one third position sensor is on said device and said relative position between said at least one first position sensor and said at least one third position sensor is known.

32. The system of claim 30, wherein said at least one third position sensor is on said scanning apparatus and said relative position between said at least one second position sensor and said at least one third position sensor is known.

33. The system of claim 25, additionally comprising:
at least one fourth position sensor at a second reference location, said at least one fourth position sensor for receiving said radiant energy and in operative communication with said data processor.

34. The system of claim 33, wherein said at least one fourth position sensor includes two receivers at a relative position from each other.

35. The system of claim 34, wherein said second reference location is a known and fixed location.

36. The system of claim 34, wherein said second reference location is unknown.

37. The system of claim 25, additionally comprising:
at least one fifth position sensor at a known and fixed position from said first reference location, said at least one fifth position sensor for receiving said radiant energy and in operative communication with said data processor.

38. The system of claim 25, wherein said at least one transmitter includes one transmitter.

39. The system of claim 25, wherein said at least one transmitter includes two transmitters, one of said transmitters being redundant.

40. A system for guiding the movements of a device toward a target viewable from an image, comprising:
at least one scanning apparatus for creating said image including said target;
at least one transmitter at a reference location for transmitting radiant energy from said reference location, said radiant energy being in the form of signals emitted so as to alternate at different measurement cycles;
at least one position sensor on said device for receiving said radiant energy and producing an output corresponding to the position of said device with respect to said reference location in space;
at least one position sensor on said at least one scanning apparatus for receiving said radiant energy and producing an output corresponding to the position of said device with respect to said reference location in space; and
a data processor in operative communication with said at least one transmitter and each of said at least one position sensors, for monitoring the accuracy of the calculated position of said device with respect to said at least one scanning apparatus.

41. The system of claim 40, wherein said position sensors are receivers.

42. The system of claim 41, wherein said receivers are magnetic and said at least one transmitter emits electromagnetic energy.

43. The system of claim 40, wherein said signals emitted so as alternate at different measurement cycles include DC or AC signals.

44. The system of claim 43, additionally comprising three independent antennae.

45. The system of claim 44, wherein said transmitter emits from each antenna at least partially sequentially during a first measurement cycle and at least partially sequentially from paired antennae during a second measurement cycle.

46. The system of claim 40, wherein said first reference location is a known and fixed location in space.

47. The system of claim 40, wherein said reference location is unknown.

48. The system of claim 40, wherein said at least one transmitter includes one transmitter.

49. The system of claim 40, wherein said at least one transmitter includes two transmitters, one of said transmitters being redundant.

50. A system for guiding a device towards a target, said target viewable from an image comprising:
imaging apparatus for creating said image;
at least one transmitter at a reference location for transmitting radiant energy from said reference location;
imaging apparatus for creating said image, said imaging apparatus including at least one position sensor for receiving said radiant energy and producing an output proportional to the position of said imaging apparatus with respect to said reference location;
an articulated arm for retaining said device to be guided, said articulated arm being calibrated to said reference location;
a data processor in operative communication with said at least one transmitter, said at least one position sensor, and said articulated arm for calculating the position of the device with respect to the imaging apparatus for displaying the position of the device with respect to the target on said image.

51. The system of claim 50, wherein, said device includes a needle.

52. The system of claim 50, wherein, said apparatus is selected from the group comprising: ultrasound apparatus, CT apparatus, MRI apparatus and X-ray apparatus.

53. The system of claim 50, additionally comprising:
at least one additional position sensor on said imaging apparatus for receiving said radiant energy, said at least one additional position sensor in operative communication with said data processor, said data processor for monitoring the accuracy of the calculated position of said device with respect to said imaging apparatus.

54. The system of claim 50 wherein,
said at least one transmitter is such that said transmitted radiant energy is in the form of signals, emitted so as to alternate at different measurement cycles; and
said data processor in operative communication with said at least one transmitter and said at least one position sensor, and said articulated arm, for monitoring the accuracy of the calculated position of said device with respect to said imaging apparatus.

55. The system of claim 50, wherein said at least one transmitter includes one transmitter.

56. The system of claim 50, wherein said at least one transmitter includes two transmitters, one of said transmitters being redundant, said data processor in operative communication with said two transmitters, said at least one position sensor, and said articulated arm, for monitoring the accuracy of the calculated position of said device with respect to said imaging apparatus.

57. A system for guiding a device towards a target, said target viewable from an image comprising:
imaging apparatus for creating said image;
at least one transmitter at a reference location for transmitting radiant energy from said reference location;
an articulated arm for retaining imaging apparatus, said articulated arm being calibrated to said reference location;
a device to be guided, said device including at least one position sensor for receiving said radiant energy and producing an output proportional to the position of said device with respect to said reference location;
said data processor in operative communication with said at least one transmitter, said at least one position sensor, and said articulated arm for calculating the position of the device with respect to the imaging apparatus for displaying the position of the device with respect to the target on said image.

58. The system of claim 57, wherein said device includes a needle.

59. The system of claim 57, additionally comprising:
at least one additional position sensor on said device for receiving said radiant energy and producing an output proportional to the position of said device with respect to said reference location; and
said at least one additional position sensor in operative communication with said data processor, said data processor for monitoring the accuracy of the calculated position of said device with respect to said imaging apparatus.

60. The system of claim 57 wherein,
said at least one transmitter is such that said transmitted radiant energy is in the form of signals, emitted so as to alternate at different measurement cycles; and
said data processor in operative communication with said at least one transmitter and said at least one position sensor, and said articulated arm, for monitoring the accuracy of the calculated position of said device with respect to said imaging apparatus.

61. The system of claim 57, wherein said at least one transmitter includes one transmitter.

62. The system of claim 57, wherein said at least one transmitter includes two transmitters, one of said transmitters being redundant, said data processor in operative communication with said two transmitters, said at least one position sensor, and said articulated arm, for monitoring the accuracy of the calculated position of said device with respect to said imaging apparatus.

63. A system for free hand directing of a guided device towards a target imaged by a scanning device comprising:
at least one transmitter for transmitting radiant energy affixed to said scanning device at a predetermined position on it;
at least one position sensor affixed to said guided device for receiving said radiant energy from said at least one transmitter and producing an output proportional to the relative position of said guided device with respect to said scanning device; and
a data processor in operative communication with said at least one transmitter and said at least one position sensor, for calculating the position of the guided device with respect to the scanning device for displaying the position of the guided device with respect to the target on said image.

64. The system of claim 63, wherein said data processor is in operative communication with said at least one transmitter and said at least one position sensor by wired or wireless links therebetween.

65. The system of claim 63, wherein said at least one transmitter and said at least one position sensor define a positioning and tracking system.

66. The system of claim 65, wherein said positioning and tracking system is magnetic.

67. The system of claim 65, wherein said positioning and tracking system is acoustic.

68. The system of claim 65, wherein said positioning and tracking system is electro-optical.

69. The system of claim 63, wherein said at least one transmitter includes one transmitter.

70. The system of claim 63, wherein said at least one transmitter includes two transmitters, one of said transmitters being redundant, said data processor in operative communication with said two transmitters and said at least one position sensor, for monitoring the accuracy of the calculated position of said guided device with respect to said scanning device.

71. The system of claim 63, wherein said at least one position sensor includes one position sensor.

72. The system of claim 63, wherein said at least one position sensor includes two position sensors, one of said position sesnors being redundant, said data processor in operative communication with said at least one transmitter and said two position sensors, for monitoring the accuracy of the calculated position of said guided device with respect to said scanning device.

73. The system of claim 63 wherein,
said at least one transmitter is such that said transmitted radiant energy is in the form of signals, emitted so as to alternate at different measurement cycles; and
said data processor in operative communication with said at least one transmitter and said at least one position sensor, for monitoring the accuracy of the calculated position of said guided device with respect to said scanning device.

74. A system for free hand directing of a guided device towards a target imaged by a scanning device comprising:
at least one transmitter for transmitting radiant energy affixed to said guided device at a predetermined position on it;
at least one position sensor affixed to said scanning device for receiving said radiant energy from said at least one transmitter and producing an output proportional to the relative position of said scanning device with respect to said guided device; and
a data processor in operative communication with said at least one transmitter and said at least one position sensor, for calculating the position of the guided device with respect to the scanning device for displaying the position of the guided device with respect to the target on said image.

75. The system of claim 74, wherein said data processor is in operative communication with said at least one transmitter and said at least one position sensor by wired or wireless links therebetween.

76. The system of claim 74, wherein said at least one transmitter and said at least one position sensor define a positioning and tracking system.

77. The system of claim 76, wherein said positioning and tracking system is magnetic.

78. The system of claim 76, wherein said positioning and tracking system is acoustic.

79. The system of claim 76, wherein said positioning and tracking system is electro-optical.

80. The system of claim 74, wherein said at least one transmitter includes one transmitter.

81. The system of claim 74, wherein said at least one transmitter includes two transmitters, one of said transmitters being redundant, said data processor in operative communication with said two transmitters and said at least one position sensor, for monitoring the accuracy of the calculated position of said guided device with respect to said scanning device.

82. The system of claim 74, wherein said at least one position sensor includes one position sensor.

83. The system of claim 74, wherein said at least one position sensor includes two position sensors, one of said position sensors being redundant, said data processor in operative communication with said at least one transmitter and said two position sensors, for monitoring the accuracy of the calculated position of said guided device with respect to said scanning device.

84. The system of claim 74 wherein,
said at least one transmitter is such that said transmitted radiant energy is in the form of signals, emitted so as to alternate at different measurement cycles; and
said data processor in operative communication with said at least one transmitter and said at least one position sensor, for monitoring the accuracy of the calculated position of said guided device with respect to said scanning device.
